(19) United States
(12) Patent Application Publication
(10) Pub. No.: US 2011/0252504 A1
(43) Pub. Date: Oct. 13, 2011

(54) Promoter From Z. Mais

(75) Inventors: Huihua Fu, Cary, NC (US); Hee-Sook Song, Raleigh, NC (US)

(73) Assignee: BASF Plant Science GmbH, Ludwigshafen (DE)

(21) Appl. No.: 13/140,502

(22) PCT Filed: Dec. 15, 2009
(86) PCT No.: PCT/EP09/67174

§ 371 Date: Jun. 17, 2011

(30) Foreign Application Priority Data
Dec. 17, 2008 (EP) 08171968.4

(51) Int. Cl. A01H 1/00; A01H 5/00; C12N 5/10; C07H 21/04; C12N 15/82

(52) U.S. Cl. 800/278; 536/24.1; 435/320.1; 435/419; 800/295

(57) ABSTRACT

The present invention is concerned with the provision of means and methods for gene expression. Specifically, it relates to a polynucleotide comprising an expression control sequence which allows for bidirectional expression of two nucleic acid of interest being operation linked thereto in opposite orientations. Furthermore, vectors, host cells, non-human transgenic organisms and methods for expressing nucleic acids of interest are provided which are based on the said polynucleotide.

The present invention is concerned with the provision of means and methods for gene expression. Specifically, it relates to a polynucleotide comprising an expression control sequence which allows for bidirectional expression of two nucleic acid of interest being operatively linked thereto in opposite orientations. Furthermore, vectors, host cells, non-human transgenic organisms and methods for expressing nucleic acids of interest are provided which are based on the said polynucleotide.

The production of transgenic plants is a fundamental technique of plant biotechnology and, thus, an indispensible prerequisite for fundamental research on plants, and for producing plants having improved, novel properties for agriculture, for increasing the quality of human foods or for producing particular chemicals or pharmaceuticals. A basic prerequisite for transgenic expression of particular genes in plants is the provision of plant-specific promoters. Various plant promoters are known. The constitutive promoters which are currently predominantly used in plants are almost exclusively viral promoters or promoters isolated from Agrobacterium such as, for example, the cauliflower mosaic virus promoter CaMV355 (Odell et al. (1985) Nature 313:810-812). The increasing complexity of the work in plant biotechnology often requires transformation with a plurality of expression constructs. Multiple use of one and the same promoter is problematic especially in plants, because the multiple presence of identical regulatory sequences may result in gene activity being switched off (silencing) (Kumpatla et al. (1998) TIBS 3:97-104; Selker (1999) Cell 97:157-160). There is thus an increasing need for novel promoters. An alternative way of dealing with this problem is the use of so-called “bidirectional” promoters, i.e. regulatory sequences which result in transcription of the upstream and downstream DNA sequences in both direction. It is possible in this case for example for target gene and marker gene to be introduced into a cell under the control of one DNA sequence.

Transgenic expression under the control of bidirectional promoters has scarcely been described to date. The production of bidirectional promoters from polar promoters for expression of nucleic acids in plants by means of fusion with further transcriptional elements has been described (Xie M (2001) Nature Biotech 19: 677-679). The 35S promoter has likewise been converted into a bidirectional promoter (Dong J Z et al. (1991) BIO/TECHNOLOGY 9: 858-863). WO 02/64804 describes the construction of a bidirectional promoter complex based on fusion of enhancer and nuclear promoter elements of various viral (CaMV 35S, CsVMV) and plant (Act2, PRb1b) sequences. US20020108142 describes a regulatory sequence from an intron of the phosphatidy-linositol transfer-like protein IV from Lotus japonicus

(PLP-IV; GenBank Acc. No.: AF367434) and the use thereof as bidirectional promoter. This intron fragment has a transcriptional activity only in the infection zone of the nodules. Other tissues, roots, leaves or flowers show no stain. Plant promoters permitting bidirectional, ubiquitous (i.e. substantially tissue-nonspecific) and constitutive expression in plants have not been disclosed to date. WO 03/006660 describes a promoter of a putative ferredoxin gene, and expression constructs, vectors and transgenic plants comprising this promoter. The isolated 836 by 5′-flanking sequence fused to the glucuronidase gene surprisingly show a constitutive expression pattern in transgenic tobacco. The sequence corresponds to a sequence segment on chromosome 4 of Arabidopsis thaliana as deposited in GenBank under the Acc. No. Z97337 (version Z97337.2; base pair 85117 to 85952; the gene starting at by 85953 is annotated with strong similarity to ferredoxin [2Fe-2S] I, Nostoc muscorum“). The activity detectable in the anthers/pollen of the closed flower buds was only weak, and in mature flowers was zero. Contrary to the prejudice derived from the literature findings against suitability of the promoter for efficient expression of selection markers (for example based on the presumed leaf specificity or the function in photosynthetic electron transport), it was possible to demonstrate highly efficient selection by combination with, for example, the kanamycin resistant gene (nptll). WO 03/006660 describes merely the use as “normal” constitutive promoter. Use as bidirectional promoter is not disclosed. In order to integrate a maximum number of genes into a plant genome via a transfer complex, it is necessary to limit the number and size of regulatory sequences for expressing transgenic nucleic acids. Promoters acting bidirectionally contribute to achieving this object. It is particularly advantageous to use a bidirectional promoter when its activities are present coordinated in the same strength and are located on a short DNA fragment. Since there is little acceptance for the use of viral sequences for expression in transgenic plants, it is advantageous to use regulatory sequences which are likewise from plants. WO2005/019459 describes a bidirectional promoter from Arabidopsis thaliana which allows for bidirectional expression in various tissues in transgenic tobacco or canola plants.

However, there is a clear need for bidirectional expression of transgenes in a timely restriced or tissue specific manner. Specifically, bidirectional expression systems allow for controlling expression of transgenes in a stoichiometric manner. Moreover, the number of expression cassettes to be introduced into an organism for heterologous gene expression can be reduced since in a bidirectional expression system, one expression control sequence governs the expression of two nucleic acids of interest.

Thus, the technical problem underlying this invention may be seen as the provision of means and methods which allow for complying with the aforementioned needs. The technical problem is solved by the embodiments characterized in the claims and herein below.

Accordingly, the present invention relates to a polynucleotide comprising an expression control sequence which, preferably, allows for bidirectional expression of two nucleic acid of interest being operatively linked thereto in opposite orientations, said expres- sion control sequence being selected from the group consisting of:
(a) an expression control sequence having a nucleic acid sequence as shown in any one of SEQ ID NOs: 1 to 3;
(b) an expression control sequence having a nucleic acid sequence which is at least 80% identical to a nucleic acid sequence shown in any one of SEQ ID NOs: 1 to 3;
(c) an expression control sequence having a nucleic acid sequence which hybridizes under stringent conditions to a nucleic acid sequence as shown in any one of SEQ ID NOs: 1 to 3;
(d) an expression control sequence having a nucleic acid sequence which hybridizes to a nucleic acid sequences located upstream of an open reading frame sequence shown in SEQ ID NO: 4;
(e) an expression control sequence having a nucleic acid sequence which hybridizes to a nucleic acid sequences located upstream of an open reading frame sequence encoding an amino acid sequence as shown in SEQ ID NO: 5;
(f) an expression control sequence having a nucleic acid sequence which hybridizes to a nucleic acid sequences located upstream of an open reading frame sequence being at least 80% identical to an open reading frame sequence as shown in SEQ ID NO: 4, wherein the open reading frame encodes a 60S acidic ribosomal protein P3;
(g) an expression control sequence having a nucleic acid sequence which hybridizes to a nucleic acid sequences located upstream of an open reading frame encoding an amino acid sequence being at least 80% identical to an amino acid sequence as shown in SEQ ID NO: 5, wherein the open reading frame encodes a 60S acidic ribosomal protein P3;
(h) an expression control sequence obtainable by 5′ genome walking or by thermal asymmetric interlaced polymerase chain reaction (TAIL-PCR) on genomic DNA from the first exon of an open reading frame sequence as shown in SEQ ID NO: 4; and
(i) an expression control sequence obtainable by 5′ genome walking or TAIL PCR on genomic DNA from the first exon of an open reading frame sequence being at least 80% identical to an open reading frame as shown in SEQ ID NO: 4, wherein the open reading frame encodes a 60S acidic ribosomal protein P3; and
(j) an expression control sequence obtainable by 5′ genome walking or TAIL PCR on genomic DNA from the first exon of an open reading frame sequence encoding an amino acid sequence being at least 80% identical to an amino acid sequence encoded by an open reading frame as shown in SEQ ID NO: 5, wherein the open reading frame encodes a 60S acidic ribosomal protein P3.

The term “polynucleotide” as used herein refers to a linear or circular nucleic acid molecule. It encompasses DNA as well as RNA molecules. The polynucleotide of the present invention is characterized in that it shall comprise an expression control sequence as defined elsewhere in this specification. In addition to the expression control sequence, the polynucleotide of the present invention, preferably, further comprises at least one nucleic acid of interest being operatively linked to the expression control sequence and/or at least one a termination sequence or transcription. Thus, the polynucleotide of the present invention, preferably, comprises an expression cassette for the expression of at least one nucleic acid of interest. More preferably, the polynucleotide comprises at least one expression cassette comprising a nucleic acid of interest and/or a terminator sequence in each orientation, i.e. the expression control sequence will be operatively linked at . Said expression cassettes are, more preferably, operatively linked to the expression both ends to at least one expression cassette, the transcription of which is governed by the said expression control sequence in opposite orientations, i.e. from one DNA strand in one direction and from the other DNA strand in the opposite direction. It will e understood that the polynucleotide, also preferably, can comprise more than one expression cassettes for each direction. Therefore, polynucleotides comprising expression cassettes with at least two, three, four or five or even more expression cassettes for nucleic acids of interest are also contemplated by the present invention. Furthermore, it will e not necessary to have equal numbers of expression cassettes for each of the two orientations, e.g., one direction may comprise two expression cassettes while the other direction of transcription from the expression control sequence may comprise only one expression cassette.

Instead of a nucleic acid of interest, the at least one expression cassette can also comprise a multiple cloning site and/or a termination sequence for transcription. In such a case, the multiple cloning site is, preferably, arranged in a manner as to allow for operative linkage of a nucleic acid to be introduced in the multiple cloning site with the expression control sequence. In addition to the aforementioned components, the polynucleotide of the present invention, preferably, could comprise components required for homologous recombination, i.e. flanking genomic sequences from a target locus. However, also contemplated is a polynucleotide which essentially consists of the said expression control sequence.

The term “expression control sequence” as used herein refers to a nucleic acid which is capable of governing the expression of another nucleic acid operatively linked thereto, e.g. a nucleic acid of interest referred to elsewhere in this specification in detail. An expression control sequence as referred to in accordance with the present invention, preferably, comprises sequence motifs which are recognized and bound by polypeptides, i.e. transcription factors. The said transcription factors shall upon binding recruit RNA polymerases, preferably, RNA polymerase I, II or III, more preferably, RNA polymerase II or III, and most preferably, RNA polymerase II. Thereby the expression of a nucleic acid operatively linked to the expression control sequence will be initiated. It is to be understood that dependent on the type of nucleic acid to be expressed, i.e. the nucleic acid of interest, expression as meant herein may comprise transcription of RNA polynucleotides from the nucleic acid sequence (as suitable for, e.g., anti-sense approaches or RNAi approaches) or may comprises transcription of RNA polynucleotides followed by translation of the said RNA polynucleotides into polypeptides (as suitable for, e.g., gene expression and recombinant polypeptide production approaches). In order to govern expression of a nucleic acid, the expression control sequence may be located immediately adjacent to the nucleic acid to be expressed, i.e. physically linked to the said nucleic acid at its 5″end. Alternatively, it may be located in physical proximity. In the latter case, however, the sequence must be located so as to allow functional interaction with the nucleic acid to be expressed. An expression control sequence referred to herein, preferably, comprises between 200 and 5,000 nucleotides in length. More preferably, it comprises between 500 and 2,500 nucleotides and, more preferably, at least 1,000 nucleotides. As mentioned before, an expression control sequence, preferably, comprises a plurality of sequence motifs which are required for transcription factor binding or for conferring a certain structure to the polynucletide comprising the expression control sequence. Sequence motifs are also sometimes referred to as cis-regulatory elements and, as meant herein, include promoter elements as well as enhancer elements. The expression control sequence of the present invention allows for bidirectional expression and, thus, comprises cis-regulatory elements which can recruit RNA polymerases at two different sites and release them in opposite directions as to enable bidirectional transcription of nucleic acids operatively linked to the said expression control sequence. Thus, one expression control sequence will be sufficient to drive transcription of two nucleic acids operatively linked thereto. Preferred expression control sequences to be included into a polynucleotide of the present invention have a nucleic acid sequence as shown in any one of SEQ ID NOs: 1 to 3.

Further preferably, an expression control sequence comprised by a polynucleotide of the present invention has a nucleic acid sequence which hybridizes to a nucleic acid sequences located upstream of an open reading frame sequence shown in any one of SEQ ID NO: 4, i.e. is a variant expression control sequence. It will be understood that expression control sequences may slightly differ in its sequences due to allelic variations. Accordingly, the present invention also contemplates an expression control sequence which can be derived from an expression control sequence as shown in any one of SEQ ID NOs: 1 to 3. Said expression control sequences are capable of hybridizing, preferably under stringent conditions, to the upstream sequences of the open reading frames shown in any one of SEQ ID NOs. 4, i.e. to the expression control sequences shown in any one of SEQ ID NOs.: 1 to 3. Stringent hybridization conditions as meant herein are, preferably, hybridization conditions in 6× sodium chloride/sodium citrate (=SSC) at approximately 45° C., followed by one or more wash steps in 0.2×SSC, 0.1% SDS at 53 to 65° C., preferably at 55° C., 56° C., 57° C., 58° C., 59° C., 60° C., 61° C., 62° C., 63° C., 64° C. or 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. Such hybridizing expression control sequences are, more preferably, at least 70%, at least 80%, at least 90%, at least 91%, at least 92%, at least 93%, at least 94% at least 95%, at least 96%, at least 97%, at least 98%, or at least 99% identical to the expression control sequences as shown in any one of SEQ ID NOs.: 1 to 3. The percent identity values are, preferably, calculated over the entire nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins 1989, CABIOS, 5: 151-153) or the programs Gap and BestFit (Needleman 1970 J. Mol. Biol. 48; 443-453 and Smith 1981, Adv. Appl. Math. 2; 482-489), which are part of the GCG software packet (Genetics Computer Group, 575 Science Drive, Madison, Wis., USA 53711 version 1991), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

Moreover, expression control sequences which allow for bidirectional expression can not only be found upstream of the aforementioned open reading frames having a nucleic acid sequence as shown in any one of SEQ ID NOs. 4. Rather, expression control sequences which allow for seed specific expression can also be found upstream of orthologous, paralogous or homologous genes (i.e. open reading frames). Thus, also preferably, an variant expression control sequence comprised by a polynucleotide of the present invention has a nucleic acid sequence which hybridizes to a nucleic acid sequences located upstream of an open reading frame sequence being at least 70%, more preferably, at least 80%, at least 90%, at least 91%, at least 92%, at least 93%, at least 94% at least 95%, at least 96%, at least 97%, at least 98%, or at least 99% identical to a sequence as shown in any one of SEQ ID NOs: 4. The said variant open reading shall encode a polypeptide having the biological activity of the corresponding polypeptide being encoded by the open reading frame shown in any one of SEQ ID NOs.: 4. In this context it should be mentioned that the open reading frame shown in SEQ ID NO: 4 encodes a polypeptide having the amino acid sequence shown in SEQ ID NO: 5 and, preferably, encodes a 60S acidic ribosomal protein P3.

Also preferably, a variant expression control sequence comprised by a polynucleotide of the present invention is (i) obtainable by 5′ genome walking or TAIL PCR from an open reading frame sequence as shown in any one of SEQ ID NOs: 4 or (ii) obtainable by 5′ genome walking or TAIL PCR from a open reading frame sequence being at least 80% identical to an open reading frame as shown in any one of SEQ ID NOs: 4. Variant expression control sequences are obtainable without further ado by the genome walking technology or by thermal asymmetric interlaced polymerase chain reaction (TAIL-PCR) which can be carried out as described in the accompanying Examples by using, e.g., commercially available kits.

Variant expression control sequences referred to in this specification for the expression control sequence shown in SEQ ID NOs: 1 to 3, preferably, comprise at least 10, at least 20, at least 30, at least 40, at least 50 or all of the sequence motifs recited in Table 3. More preferably, the variant expression control sequence comprises the sequence motifs shown in any one of SEQ ID NOs: 54 to 76.

Also preferably, the expression control sequence comprised by the polynucleotide of the present invention allows for a tissue specific expression. Tissues in which the expression control sequence allows for bidirectional specific expression are the following indicated tissues and cells: 1) roots and leafs at 5-leaf stage, 2) stem at V-7 stage, 3) Leaves, husk, and silk at flowering stage (at the first emergence of silk), 4) Spikelets/Tassel at pollination, 5) Ear or Kernels at 5, 10, 15, 20, and 25 days after pollination.

More preferably, specific expression in the forward direction of the expression control sequence of the present invention is in the seed, preferably, whole seed, and the stem. Also more preferably, specific expression in the reverse direction of the expression control sequence of the present invention can be seen in leaf and root.

The term “specific” as used herein means that the nucleic acids of interest being operatively linked to the expression control sequence referred to herein will be predominantly expressed in the indicated tissues or cells when present in a plant. A predominant expression as meant herein is characterized by a statistically significantly higher amount of detectable transcription in the said tissue or cells with respect to other plant tissues. A statistically significant higher amount of transcription is, preferably, an amount being at least two-fold, three-fold, four-fold, five-fold, ten-fold, hundred-fold, five hundred-fold or thousand-fold the amount found in at least one of the other tissues with detectable transcription. Alternatively, it is an expression in the indicated tissue or cell whereby the amount of transcription in other tissues or cells is less than 1%, 2%, 3%, 4% or, most preferably, 5% of the overall (whole plant) amount of expression. The amount of transcription directly correlates to the amount of transcripts (i.e. RNA) or polypeptides encoded by the transcripts present in a cell or tissue. Suitable techniques for measuring transcription either based on RNA or polypeptides are well known in the art. Tissue or cell specificity alternatively and, preferably in addition to the above, means that the expression is restricted or almost restricted to the indicated tissue or cells, i.e. there is essentially no detectable transcription in other tissues. Almost restricted as meant herein means that unspecific expression is detectable in less than ten, less than five, less than four, less than three, less than two or one other tissue(s). Tissue or cell specific expression as used herein includes expression in the indicated tissue or cells as well as in precursor tissue or cells in the developing embryo.

An expression control sequences can be tested for tissue or cell specific expression by determining the expression pattern of a nucleic acid of interest, e.g., a nucleic acid encoding a reporter protein, such as GFP, in a transgenic plant. Transgenic plants can be generated by techniques well known to the person skilled in the art and as discussed elsewhere in this specification. The aforementioned amounts or expression pattern are, preferably, determined by Northern Blot or in situ hybridization techniques as described in WO 02/102970. Preferred expression pattern for the expression control sequences according to the present invention are shown in the Figure or described in the accompanying Examples, below.

The term “nucleic acid of interest” refers to a nucleic acid which shall be expressed under the control of the expression control sequence referred to herein. Preferably, a nucleic acid of interest encodes a polypeptide the presence of which is desired in a cell or non-human organism as referred to herein and, in particular, in a plant seed. Such a polypeptide may be an enzyme which is required for the synthesis of seed storage compounds or may be a seed storage protein. It is to be understood that if the nucleic acid of interest encodes a polypeptide, transcription of the nucleic acid in RNA and translation of the transcribed RNA into the polypeptide may be required. A nucleic acid of interest, also preferably, includes biologically active RNA molecules and, more preferably, antisense RNAs, ribozymes, micro RNAs or siRNAs. Said biologically active RNA molecules can be used to modify the amount of a target polypeptide present in a cell or non-human organism. For example, an undesired enzymatic activity in a seed can be reduced due to the seed specific expression of an antisense RNAs, ribozymes, micro RNAs or siRNAs. The underlying biological principles of action of the aforementioned biologically active RNA molecules are well known in the art. Moreover, the person skilled in the art is well aware of how to obtain nucleic acids which encode such biologically active RNA molecules. It is to be understood that the biologically active RNA molecules may be directly obtained by transcription of the nucleic acid of interest, i.e. without translation into a polypeptide. Preferably, at least one nucleic acid of interest to be expressed under the control of the expression control sequence of the present invention is heterologous in relation to said expression control sequence, i.e. it is not naturally under the control thereof, but said control has been produced in a non-natural manner (for example by genetic engineering processes).

The term “operatively linked” as used herein means that the expression control sequence of the present invention and a nucleic acid of interest, are linked so that the expression can be governed by the said expression control sequence, i.e. the expression control sequence shall be functionally linked to the said nucleic acid sequence to be expressed. Accordingly, the expression control sequence and, the nucleic acid sequence to be expressed may be physically linked to each other, e.g., by inserting the expression control sequence at the 5′end of the nucleic acid sequence to be expressed. Alternatively, the expression control sequence and the nucleic acid to be expressed may be merely in physical proximity so that the expression control sequence is capable of governing the expression of at least one nucleic acid sequence of interest. The expression control sequence and the nucleic acid to be expressed are, preferably, separated by not more than 500 bp, 300 bp, 100 bp, 80 bp, 60 bp, 40 bp, 20 bp, 10 by or 5 bp. For the bidirectional expression control sequence of the present invention it is to e understood that the above applies for both of the operatively the nucleic acids of interest. It will be understood that non-essential sequences of one of the expression control sequence of the invention can be deleted without significantly impairing the properties mentioned. Delimitation of the expression control sequence to particular essential regulatory regions can also be undertaken with the aid of a computer program such as the PLACE program (“Plant Cis-acting Regulatory DNA Elements”) (Higo K et al. (1999) Nucleic Acids Res 27:1, 297-300) or the BIOBASE database “Transfac” (Biologische Datenbanken GmbH, Braunschweig). Processes for mutagenizing nucleic acid sequences are known to the skilled worker and include by way of example the use of oligonucleotides having one or more mutations compared with the region to be mutated (e.g. within the framework of a site-specific mutagenesis). Primers having approximately 15 to approximately 75 nucleotides or more are typically employed, with preferably about 10 to about 25 or more nucleotide residues being located on both sides of the sequence to be modified. Details and procedure for said mutagenesis processes are familiar to the skilled worker (Kunkel et al. (1987) Methods Enzymol 154:367-382; Tomic et al. (1990) Nucl Acids Res 12:1656; Upender et al. (1995) Biotechniques 18(1):29-30; U.S. Pat. No. 4,237,224). A mutagenesis can also be achieved by treatment of, for example, vectors comprising one of the nucleic acid sequences of the invention with mutagenizing agents such as hydroxylamine.

Advantageously, it has been found in the studies underlying the present invention that bidirectional expression of two nucleic acids of interest can be achieved by expressing said nucleic acids of interest under the control of an expression control sequence from maize or a variant expression control sequence as specified above. The expression control sequences provided by the present invention allow for a reliable bidirectional expression of nucleic acids of interest. Thanks to the present invention, it is possible to (i) specifically manipulate biochemical processes in specific tissues, e.g., by expressing heterologous enzymes or biologically active RNAs, or (ii) to produce heterologous proteins in said tissues, or (iii) to provide nucleic acids of interest in a stoichiometric ratio. In principle, the present invention contemplates the use of the polynucleotide, the vector, the host cell or the non-human transgenic organism for the expression of a nucleic acid of interest. The invention makes it possible to increase the number of transcription units with a reduced number of promoter sequences. In the case of translation fusions it is also possible to regulate more than two proteins. A particular advantage of this invention is that the expression of these multiple transgenes takes place simultaneously and synchronously under the control of the bidirectional promoter. The promoter is particularly suitable for coordinating expression of nucleic acids. Thus, it is possible to express simultaneously: (i) target protein and selection marker or reporter protein, ii) selection marker and reporter protein, iii) two target proteins, e.g. from the same metabolic pathway iii) sense and antisense RNA, iv) various proteins for defense against pathogens, and many more, and v) bring about improved effects in the plants.

The present invention also relates to a vector comprising the polynucleotide of the present invention.

The term “vector”, preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotides of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, natural competence, carbon-based clusters, chemically mediated transfer, electroporation or particle bombardment (e.g., “gene-gun”). Suitable methods for the transformation or transfection of host cells, including plant cells, can be found in Sambrook et al. (Molecular Cloning: A Laboratory Manual, 2^{nd} ed., Cold Spring Harbor Laboratory, Cold Spring Harbor Laboratory Press, Cold Spring Harbor, N.Y., 1989) and other laboratory manuals, such as Methods in Molecular Biology, 1995, Vol. 44, Agrobacterium protocols, Ed.: Gartland and Davey, Humana Press, Totowa, N.J. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells.

Preferably, the vector referred to herein is suitable as a cloning vector, i.e. replicable in microbial systems. Such vectors ensure efficient cloning in bacteria and, preferably, yeasts or fungi and make possible the stable transformation of plants. Those which must be mentioned are, in particular, various binary and co-integrated vector systems which are suitable for the T-DNA-mediated transformation. Such vector systems are, as a rule, characterized in that they contain at least the vir genes, which are required for the Agrobacterium-mediated transformation, and the sequences which delimit the T-DNA (T-DNA border). These vector systems, preferably, also comprise further cis-regulatory regions such as promoters and terminators and/or selection markers with which suitable transformed host cells or organisms can be identified. While co-integrated vector systems have vir genes and T-DNA sequences arranged on the same vector, binary systems are based on at least two vectors, one of which bears vir genes, but no T-DNA, while a second one bears T-DNA, but no vir gene. As a consequence, the last-mentioned vectors are relatively small, easy to manipulate and can be replicated both in E. coli and in Agrobacterium. These binary vectors include vectors from the pBIB-HYG, pPZP, pBecks, pGreen series. Preferably used in accordance with the invention are Bin19, pBI101, pBinAR, pGPTV, pSUN and pCAMBIA. An overview of binary vectors and their use can be found in Hellens et al, Trends in Plant Science (2000) 5, 446-451. Furthermore, by using appropriate cloning vectors, the polynucleotide of the invention can be introduced into host cells or organisms such as plants or animals and, thus, be used in the transformation of plants, such as those which are published, and cited, in: Plant Molecular Biology and Biotechnology (CRC Press, Boca Raton, Fla.), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205-225.

More preferably, the vector of the present invention is an expression vector. In such an expression vector, the polynucleotide comprises an expression cassette as specified above allowing for expression in eukaryotic cells or isolated fractions thereof. An expression vector may, in addition to the polynucleotide of the invention, also comprise further regulatory elements including transcriptional as well as translational enhancers. Preferably, the expression vector is also a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

Suitable expression vector backbones are, preferably, derived from expression vectors known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX (Pharmacia Biotech Inc; Smith, D. B., and Johnson, K. S. (1988) Gene 67:31-40), pMAL (New England Biolabs, Beverly, Mass.) and pRIT5 (Pharmacia, Piscataway, N.J.), where glutathione S-transferase (GST), maltose E-binding protein and protein A, respectively, are fused with the nucleic acid of interest encoding a protein to be expressed. The target gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a coexpressed viral RNA polymerase (T7 gn1). This viral polymerase is provided by the host strains BL21 (DE3) or HMS174 (DE3) from a resident λ-prophage which harbors a T7 gn1 gene under the transcriptional control of the lacUV 5 promoter. Examples of vectors for expression in the yeast S. cerevisiae comprise pYepSecl (Baldari et al. (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.). Vectors and processes for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells (for example Sf9 cells) comprise the pAc series (Smith et al. (1983) Mol. Cell Biol. 3:2156-2165) and the pVL series (Lucklow and Summers (1989) Virology 170:31-39).

The polynucleotides of the present invention can be used for expression of a nucleic acid of interest in single-cell plant cells (such as algae), see Falciatore et al., 1999, Marine Biotechnology 1 (3):239-251 and the references cited therein, and plant cells from higher plants (for example Spermatophytes, such as arable crops) by using plant expression vectors. Examples of plant expression vectors comprise those which are described in detail in: Becker, D., Kemper, E., Schell, J., and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721; Vectors for Gene Transfer in Higher Plants; in: Transgenic Plants, Vol. 1, Engineering and Utilization, Ed.: Kung and R. Wu, Academic Press, 1993, p. 15-38. A plant expression cassette, preferably, comprises regulatory sequences which are capable of controlling the gene expression in plant cells and which are functionally linked so that each sequence can fulfill its function, such as transcriptional termination, for example polyadenylation signals. Preferred polyadenylation signals are those which are derived from Agrobacterium tumefaciens T-DNA, such as the gene 3 of the Ti plasmid pTiACH5, which is known as octopine synthase (Gielen et al., EMBO J. 3 (1984) 835 et seq.) or functional equivalents of these, but all other terminators which are functionally active in plants are also suitable.

Since plant gene expression is very often not limited to transcriptional levels, a plant expression cassette preferably comprises other functionally linked sequences such as translation enhancers, for example the overdrive sequence, which comprises the 5′-untranslated tobacco mosaic virus leader sequence, which increases the protein/RNA ratio (Gallie et al., 1987, Nucl. Acids Research 15:8693-8711). Other preferred sequences for the use in functional linkage in plant gene expression cassettes are targeting sequences which are required for targeting the gene product into its relevant cell compartment (for a review, see Kermode, Crit. Rev. Plant Sci. 15, 4 (1996) 285-423 and references cited therein), for example into the vacuole, the nucleus, all types of plastids, such as amyloplasts, chloroplasts, chromoplasts, the extracellular space, the mitochondria, the endoplasmic reticulum, oil bodies, peroxisomes and other compartments of plant cells.

The abovementioned vectors are only a small overview of vectors to be used in accordance with the present invention. Further vectors are known to the skilled worker and are described, for example, in: Cloning Vectors (Ed., Pouwels, P. H., et al., Elsevier, Amsterdam-New York-Oxford, 1985, ISBN 0 444 904018). For further suitable expression systems for prokaryotic and eukaryotic cells see the chapters 16 and 17 of Sambrook, J., Fritsch, E. F., and Maniatis, T., Molecular Cloning: A Laboratory Manual, 2^{nd} edition, Cold Spring Harbor Laboratory, Cold Spring Harbor Laboratory Press, Cold Spring Harbor, N.Y., 1989.

The present invention also contemplates a host cell comprising the polynucleotide or the vector of the present invention.

Host cells are primary cells or cell lines derived from multicellular organisms such as plants or animals. Furthermore, host cells encompass prokaryotic or eukaryotic single cell organisms (also referred to as micro-organisms). Primary cells or cell lines to be used as host cells in accordance with the present invention may be derived from the multicellular organisms referred to below. Host cells which can be exploited are furthermore mentioned in: Goeddel, Gene Expression Technology: Methods in Enzymology 185, Academic Press, San Diego, Calif. (1990). Specific expression strains which can be used, for example those with a lower protease activity, are described in: Gottesman, S., Gene Expression Technology: Methods in Enzymology 185, Academic Press, San Diego, Calif. (1990) 119-128. These include plant cells and certain tissues, organs and parts of plants in all their phenotypic forms such as anthers, fibers, root hairs, stalks, embryos, calli, cotelydons, petioles, harvested material, plant tissue, reproductive tissue and cell cultures which are derived from the actual transgenic plant and/or can be used for bringing about the transgenic plant. Preferably, the host cells may be obtained from plants. More preferably, oil crops are envisaged which comprise large amounts of lipid compounds, such as oilseed rape, evening primrose, hemp, thistle, peanut, canola, linseed, soybean, safflower, sunflower, borage, or plants such as maize, wheat, rye, oats, triticale, rice, barley, cotton, cassava, pepper, Tagetes, Solanaceae plants such as potato, tobacco, eggplant and tomato, Vicia species, pea, alfalfa, bushy plants (coffee, cacao, tea), Salix species, trees (oil palm, coconut) and perennial grasses and fodder crops. Especially preferred plants according to the invention are oil crops such as soybean, peanut, oilseed rape, canola, linseed, hemp, evening primrose, sunflower, safflower, trees (oil palm, coconut). Suitable methods for obtaining host cells from the multicellular organisms referred to below as well as conditions for culturing these cells are well known in the art.

The micro-organisms are, preferably, bacteria or fungi including yeasts. Preferred fungi to be used in accordance with the present invention are selected from the group of the families Chaetomiaceae, Choanephoraceae, Cryptococcaceae, Cunninghamellaceae, Demetiaceae, Moniliaceae, Mortierellaceae, Mucoraceae, Pythiaceae, Sacharomycetaceae, Saprolegniaceae, Schizosacharomycetaceae, Sodariaceae or Tuberculariaceae. Further preferred micro-organisms are selected from the group: Choanephoraceae such as the genera Blakeslea, Choanephora, for example the genera and species Blakeslea trispora, Choanephora cucurbitarum, Choanephora infundibulifera var. cucurbitarum, Mortierellaceae, such as the genus Mortierella, for example the genera and species Mortierella isabellina, Mortierella polycephala, Mortierella ramanniana, Mortierella vinacea, Mortierella zonata, Pythiaceae such as the genera Phytium, Phytophthora for example the genera and species Pythium debaryanum, Pythium intermedium, Pythium irregulare, Pythium megalacanthum, Pythium paroecandrum, Pythium sylvaticum, Pythium ultimum, Phytophthora cactorum, Phytophthora cinnamomi, Phytophthora citricola, Phytophthora citrophthora, Phytophthora cryptogea, Phytophthora drechsleri, Phytophthora erythroseptica, Phytophthora lateralis, Phytophthora megasperma, Phytophthora nicotianae, Phytophthora nicotianae var. parasitica, Phytophthora palmivora, Phytophthora parasitica, Phytophthora syringae, Saccharomycetaceae such as the genera Hansenula, Pichia, Saccharomyces, Saccharomycodes, Yarrowia for example the genera and species Hansenula anomala, Hansenula califonica, Hansenula canadensis, Hansenula capsulata, Hansenula ciferrii, Hansenula glucozyma, Hansenula henricii, Hansenula holstii, Hansenula minuta, Hansenula nonfermentans, Hansenula philodendri, Hansenula polymorpha, Hansenula saturnus, Hansenula subpelliculosa, Hansenula wickerhamii, Hansenula wingei, Pichia alcoholophila, Pichia angusta, Pichia anomala, Pichia bispora, Pichia burtonii, Pichia canadensis, Pichia capsulata, Pichia carsonii, Pichia cellobiosa, Pichia ciferrii, Pichia farinosa, Pichia fermentans, Pichia finlandica, Pichia glucozyma, Pichia guilliermondii, Pichia haplophila, Pichia henricii, Pichia holstii, Pichia jadinil, Pichia lindnerii, Pichia membranaefaciens, Pichia methanolica, Pichia minuta var. minuta, Pichia minuta var. nonfermentans, Pichia norvegensis, Pichia ohmeri, Pichia pastoris, Pichia philodendri, Pichia pini, Pichia polymorpha, Pichia quercuum, Pichia rhodanensis, Pichia sargentensis, Pichia stipitis, Pichia strasburgensis, Pichia subpelliculosa, Pichia toletana, Pichia trehalophila, Pichia vini, Pichia xylosa, Saccharomyces aceti, Saccharomyces bailii, Saccharomyces bayanus, Saccharomyces bisporus, Saccharomyces capensis, Saccharomyces carlsbergensis, Saccharomyces cerevisiae, Saccharomyces cerevisiae var. ellipsoideus, Saccharomyces chevalieri, Saccharomyces delbrueckii, Saccharomyces diastaticus, Saccharomyces drosophilarum, Saccharomyces elegans, Saccharomyces ellipsoideus, Saccharomyces fermentati, Saccharomyces florentinus, Saccharomyces fragilis, Saccharomyces heterogenicus, Saccharomyces hienipiensis, Saccharomyces inusitatus, Saccharomyces italicus, Saccharomyces kluyveri, Saccharomyces krusei, Saccharomyces lactis, Saccharomyces marxianus, Saccharomyces microellipsoides, Saccharomyces montanus, Saccharomyces norbensis, Saccharomyces oleaceus, Saccharomyces paradoxus, Saccharomyces pastorianus, Saccharomyces pretoriensis, Saccharomyces rosei, Saccharomyces rouxii, Saccharomyces uvarum, Saccharomycodes ludwigii, Yarrowia lipolytica, Schizosacharomycetaceae such as the genera Schizosaccharomyces e.g. the species Schizosaccharomyces japonicus var. japonicus, Schizosaccharomyces japonicus var. versatilis, Schizosaccharomyces malidevorans, Schizosaccharomyces octosporus, Schizosaccharomyces pombe var. malidevorans, Schizosaccharomyces pombe var. pombe, Thraustochytriaceae such as the genera Althornia, Aplanochytrium, Japonochytrium, Schizochytrium, Thraustochytrium e.g. the species Schizochytrium aggregatum, Schizochytrium limacinum, Schizochytrium mangrovei, Schizochytrium minutum, Schizochytrium octosporum, Thraustochytrium aggregatum, Thraustochytrium amoeboideum, Thraustochytrium antacticum, Thraustochytrium arudimentale, Thraustochytrium aureum, Thraustochytrium benthicola, Thraustochytrium globosum, Thraustochytrium indicum, Thraustochytrium kerguelense, Thraustochytrium kinnei, Thraustochytrium motivum, Thraustochytrium multirudimentale, Thraustochytrium pachydermum, Thraustochytrium proliferum, Thraustochytrium roseum, Thraustochytrium Thraustochytrium striatum or Thraustochytrium visurgense. Further preferred micro-organisms are bacteria selected from the group of the families Bacillaceae, Enterobacteriacae or Rhizobiaceae. Examples of such micro-organisms may be selected from the group: Bacillaceae such as the genera Bacillus for example the genera and species Bacillus acidocaldarius, Bacillus acidoterrestris, Bacillus alcalophilus, Bacillus amyloliquefaciens, Bacillus amylolyticus, Bacillus brevis, Bacillus cereus, Bacillus circulans, Bacillus coagulans, Bacillus sphaericus subsp. fusiformis, Bacillus galactophilus, Bacillus globisporus, Bacillus globisporus subsp. marinus, Bacillus halophilus, Bacillus lentimorbus, Bacillus lentus, Bacillus licheniformis, Bacillus megaterium, Bacillus polymyxa, Bacillus psychrosaccharolyticus, Bacillus pumilus, Bacillus sphaericus, Bacillus subtilis subsp. spizizenii, Bacillus subtilis subsp. subtilis or Bacillus thuringiensis; Enterobacteriacae such as the genera Citrobacter, Edwardsiella, Enterobacter, Erwinia, Escherichia, Klebsiella, Salmonella or Serratia for example the genera and species Citrobacter amalonaticus, Citrobacter diversus, Citrobacter freundii, Citrobacter genomospecies, Citrobacter gillenii, Citrobacter intermedium, Citrobacter koseri, Citrobacter murliniae, Citrobacter sp., Edwardsiella hoshinae, Edwardsiella ictaluri, Edwardsiella tarda, Erwinia alni, Erwinia amylovora, Erwinia ananatis, Erwinia aphidicola, Erwinia billingiae, Erwinia cacticida, Erwinia cancerogena, Erwinia carnegieana, Erwinia carotovora subsp. atroseptica, Erwinia carotovora subsp. betavasculorum, Erwinia carotovora subsp. odorifera, Erwinia carotovora subsp. wasabiae, Erwinia chrysanthemi, Erwinia cypripedii, Erwinia dissolvens, Erwinia herbicola, Erwinia mallotivora, Erwinia milletiae, Erwinia nigrifluens, Erwinia nimipressuralis, Erwinia persicina, Erwinia psidii, Erwinia pyrifoliae, Erwinia quercina, Erwinia rhapontici, Erwinia rubrifaciens, Erwinia salicis, Erwinia stewartii, Erwinia tracheiphila, Erwinia uredovora, Escherichia adecarboxylata, Escherichia anindolica, Escherichia aurescens, Escherichia blattae, Escherichia coli, Escherichia coli var. communion, Escherichia coli-mutabile, Escherichia fergusonii, Escherichia hermannii, Escherichia sp., Escherichia vulneris, Klebsiella aerogenes, Klebsiella edwardsii subsp. atlantae, Klebsiella ornithinolytica, Klebsiella oxytoca, Klebsiella planticola, Klebsiella pneumoniae, Klebsiella pneumoniae subsp. pneumoniae, Klebsiella sp., Klebsiella terrigena, Klebsiella trevisanii, Salmonella abony, Salmonella arizonae, Salmonella bongori, Salmonella choleraesuis subsp. arizonae, Salmonella choleraesuis subsp. bongori, Salmonella choleraesuis subsp. cholereasuis, Salmonella choleraesuis subsp. diarizonae, Salmonella choleraesuis subsp. houtenae, Salmonella choleraesuis subsp. indica, Salmonella choleraesuis subsp. salamae, Salmonella daressalaam, Salmonella enterica subsp. houtenae, Salmonella enterica subsp. salamae, Salmonella enteritidis, Salmonella gallinarum, Salmonella heidelberg, Salmonella panama, Salmonella senftenberg, Salmonella typhimurium, Serratia entomophila, Serratia ficaria, Serratia fonticola, Serratia grimesii, Serratia liquefaciens, Serratia marcescens, Serratia marcescens subsp. marcescens, Serratia marinorubra, Serratia odorifera, Serratia plymouthensis, Serratia plymuthica, Serratia proteamaculans, Serratia proteamaculans subsp. quinovora, Serratia quinivorans or Serratia rubidaea; Rhizobiaceae such as the genera Agrobacterium, Carbophilus, Chelatobacter, Ensifer, Rhizobium, Sinorhizobium for example the genera and species Agrobacterium atlanticum, Agrobacterium ferrugineum, Agrobacterium gelatinovorum, Agrobacterium larrymoorei, Agrobacterium meteori, Agrobacterium radiobacter, Agrobacterium rhizogenes, Agrobacterium rubi, Agrobacterium stellulatum, Agrobacterium tumefaciens, Agrobacterium vitis, Carbophilus carboxidus, Chelatobacter heintzii, Ensifer adhaerens, Ensifer arboris, Ensifer fredii, Ensifer kostiensis, Ensifer kummerowiae, Ensifer medicae, Ensifer meliloti, Ensifer saheli, Ensifer terangae, Ensifer xinjiangensis, Rhizobium ciceri Rhizobium etli, Rhizobium fredii, Rhizobium galegae, Rhizobium gallicum, Rhizobium giardinii, Rhizobium hainanense, Rhizobium huakuii, Rhizobium huautlense, Rhizobium indigoferae, Rhizobium japonicum, Rhizobium leguminosarum, Rhizobium loessense, Rhizobium loti, Rhizobium lupini, Rhizobium mediterraneum, Rhizobium meliloti, Rhizobium mongolense, Rhizobium phaseoli, Rhizobium radiobacter, Rhizobium rhizogenes, Rhizobium rubi, Rhizobium sullae, Rhizobium tianshanense, Rhizobium trifolii, Rhizobium tropici, Rhizobium undicola, Rhizobium vitis, Sinorhizobium adhaerens, Sinorhizobium arboris, Sinorhizobium fredii, Sinorhizobium kostiense, Sinorhizobium kummerowiae, Sinorhizobium medicae, Sinorhizobium meliloti, Sinorhizobium morelense, Sinorhizobium saheli or Sinorhizobium xinjiangense.

How to culture the aforementioned micro-organisms is well known to the person skilled in the art.

The present invention also relates to a non-human transgenic organism, preferably a plant or seed thereof, comprising the polynucleotide or the vector of the present invention.

The term “non-human transgenic organism”, preferably, relates to a plant, a plant seed, a non-human animal or a multicellular micro-organism. The polynucleotide or vector may be present in the cytoplasm of the organism or may be incorporated into the genome either heterologous or by homologous recombination. Host cells, in particular those obtained from plants or animals, may be introduced into a developing embryo in order to obtain mosaic or chimeric organisms, i.e. non-human transgenic organisms comprising the host cells of the present invention. Suitable transgenic organisms are, preferably, all organisms which are suitable for the expression of recombinant genes.

Preferred plants to be used for making non-human transgenic organisms according to the present invention are all dicotyledonous or monocotyledonous plants, algae or mosses. Advantageous plants are selected from the group of the plant families Adelotheciaceae, Anacardiaceae, Asteraceae, Apiaceae, Betulaceae, Boraginaceae, Brassicaceae, Bromeliaceae, Caricaceae, Cannabaceae, Convolvulaceae, Chenopo- diaceae, Crypthecodiniaceae, Cucurbitaceae, Ditrichaceae, Elaeagnaceae, Ericaceae, Euphorbiaceae, Fabaceae, Geraniaceae, Gramineae, Juglandaceae, Lauraceae, Leguminosae, Linaceae, Prasinophyceae or vegetable plants or ornamentals such as Tagetes. Examples which may be mentioned are the following plants selected from the group consisting of: Adelotheciaceae such as the genera Physcomitrella, such as the genus and species Physcomitrella patens, Anacardiaceae such as the genera Pistacia, Mangifera, Anacardium, for example the genus and species Pistacia vera [pistachio], Mangifer indica [mango] or Anacardium occidentale [cashew], Asteraceae, such as the genera Calendula, Carthamus, Centaurea, Cichorium, Cynara, Helianthus, Lactuca, Locusta, Tagetes, Valeriana, for example the genus and species Calendula officinalis [common marigold] Carthamus tinctorius [safflower], Centaurea cyanus [cornflower], Cichorium intybus [chicory], Cynara scolymus [artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [salad vegetables], Tagetes lucida, Tagetes erecta or Tagetes tenuifolia [african or french marigold], Apiaceae, such as the genus Daucus, for example the genus and species Daucus carota [carrot], Betulaceae, such as the genus Corylus, for example the genera and species Corylus avellana or Corylus colurna [hazelnut], Boraginaceae, such as the genus Borago, for example the genus and species Borago officinalis [borage], Brassicaceae, such as the genera Brassica, Melanosinapis, Sinapis, Arabadopsis, for example the genera and species Brassica napus, Brassica rapa ssp. [oilseed rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana, Bromeliaceae, such as the genera Anana, Bromelia (pineapple), for example the genera and species Anana comosus, Ananas ananas or Bromelia comosa [pineapple], Caricaceae, such as the genus Carica, such as the genus and species Carica papaya [pawpaw], Cannabaceae, such as the genus Cannabis, such as the genus and species Cannabis sativa [hemp], Convolvulaceae, such as the genera Ipomea, Convolvulus, for example the genera and species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, batate], Chenopodiaceae, such as the genus Beta, such as the genera and species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugarbeet], Crypthecodiniaceae, such as the genus Crypthecodinium, for example the genus and species Cryptecodinium cohnii, Cucurbitaceae, such as the genus Cucurbita, for example the genera and species Cucurbita maxima, Cucurbita mixta, Cucurbita pepo or Cucurbita moschata [pumpkin/squash], Cymbellaceae such as the genera Amphora, Cymbella, Okedenia, Phaeodactylum, Reimeria, for example the genus and species Phaeodactylum tricornutum, Ditrichaceae such as the genera Ditrichaceae, Astomiopsis, Ceratodon, Chrysoblastella, Ditrichum, Distichium, Eccremidium, Lophidion, Philibertiella, Pleuridium, Saelania, Trichodon, Skottsbergia, for example the genera and species Ceratodon antarcticus, Ceratodon columbiae, Ceratodon heterophyllus, Ceratodon purpureus, Ceratodon purpureus, Ceratodon purpureus ssp. convolutus, Ceratodon, purpureus spp. stenocarpus, Ceratodon purpureus var. rotundifolius, Ceratodon ratodon, Ceratodon stenocarpus, Chrysoblastella chilensis, Ditrichum ambiguum, Ditrichum brevisetum, Ditrichum crispatissimum, Ditrichum difficile, Ditrichum falcifolium, Ditrichum flexicaule, Ditrichum giganteum, Ditrichum heteromallum, Ditrichum lineare, Ditrichum lineare, Ditrichum montanum, Ditrichum montanum, Ditrichum pallidum, Ditrichum punctulatum, Ditrichum pusillum, Ditrichum pusillum var. tortile, Ditrichum rhynchostegium, Ditrichum schimperi, Ditrichum tortile, Distichium capillaceum, Distichium hagenii, Distichium inclinatum, Distichium macounii, Eccremidium floridanum, Eccremidium whiteleggei, Lophidion strictus, Pleuridium acuminatum, Pleuridium alternifolium, Pleuridium holdridgei, Pleuridium mexicanum, Pleuridium ravenelii, Pleuridium subulatum, Saelania glaucescens, Trichodon borealis, Trichodon cylindricus or Trichodon cylindricus var. oblongus, Elaeagnaceae such as the genus Elaeagnus, for example the genus and species Olea europaea [olive], Ericaceae such as the genus Kalmia, for example the genera and species Kalmia latifolia, Kalmia angustifolia, Kalmia microphylla, Kalmia polifolia, Kalmia occidentalis, Cistus chamaerhodendros or Kalmia lucida [mountain laurel], Euphorbiaceae such as the genera Manihot, Janipha, Jatropha, Ricinus, for example the genera and species Manihot utilissima, Janipha manihot, Jatropha manihot, Manihot aipil, Manihot dulcis, Manihot manihot, Manihot melanobasis, Manihot esculenta [manihot] or Ricinus communis [castor-oil plant], Fabaceae such as the genera Pisum, Albizia, Cathormion, Feuillea, Inga, Pithecolobium, Acacia, Mimosa, Medicajo, Glycine, Dolichos, Phaseolus, Soja, for example the genera and species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [silk tree], Medicago sativa, Medicago falcata, Medicago varia [alfalfa], Glycine max Dolichos soja, Glycine gracilis, Glycine hispida, Phaseolus max, Soja hispida or Soja max [soybean], Funariaceae such as the genera Aphanorrhegma, Entosthodon, Funaria, Physcomitrella, Physcomitrium, for example the genera and species Aphanorrhegma serratum, Entosthodon attenuatus, Entosthodon bolanderi, Entosthodon bonplandii, Entosthodon californicus, Entosthodon drummondii, Entosthodon jamesonii, Entosthodon leibergii, Entosthodon neoscoticus, Entosthodon rubrisetus, Entosthodon spathulifolius, Entosthodon tucsoni, Funaria americana, Funaria bolanderi, Funaria calcarea, Funaria californica, Funaria calvescens, Funaria convoluta, Funaria flavicans, Funaria groutiana, Funaria hygrometrica, Funaria hygrometrica var. arctica, Funaria hygrometrica var. calvescens, Funaria hygrometrica var. convoluta, Funaria hygrometrica var. muralis, Funaria hygrometrica var. utahensis, Funaria microstoma, Funaria microstoma var. obtusifolia, Funaria muhlenbergii, Funaria orcuttii, Funaria piano-convexa, Funaria polaris, Funaria ravenelii, Funaria rubriseta, Funaria serrata, Funaria sonorae, Funaria sublimbatus, Funaria tucsoni, Physcomitrella californica, Physcomitrella patens, Physcomitrella reader, Physcomitrium australe, Physcomitrium califomicum, Physcomitrium collenchymatum, Physcomitrium coloradense, Physcomitrium cupuliferum, Physcomitrium drummondii, Physcomitrium eurystomum, Physcomitrium flexifolium, Physcomitrium hookeri, Physcomitrium hookeri var. serratum, Physcomitrium immersum, Physcomitrium kellermanii, Physcomitrium megalocarpum, Physcomitrium pyriforme, Physcomitrium pyriforme var. serratum, Physcomitrium rufipes, Physcomitrium sandbergii, Physcomitrium subsphaericum, Physcomitrium washingtoniense, Geraniaceae, such as the genera Pelargonium, Cocos, Oleum, for example the genera and species Cocos nucifera, Pelargonium grossularioides or Oleum cocois [coconut], Gramineae, such as the genus Saccharum, for example the genus and species Saccharum officinarum, Juglandaceae, such as the genera Juglans, Wallia, for example the genera and species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut], Lauraceae, such as the genera Persea, Laurus, for example the genera and species Laurus nobilis [bay], Persea americana, Persea gratissima or Persea persea [avocado], Leguminosae, such as the genus Arachis, for example the genus and species Arachis hypogaea [peanut], Linaceae, such as the genera Linum, Adenolinum, for example the genera and species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [linseed], Lythrarieae, such as the genus Punica, for example the genus and species Punica granatum [pomegranate], Malvaceae, such as the genus Gossypium, for example the genera and species Gossypium hirsutum, Gossypium arboreum, Gossypium barbadense, Gossypium herbaceum or Gossypium thurberi [cotton], Marchantiaceae, such as the genus Marchantia, for example the genera and species Marchantia berteroana, Marchantia foliacea, Marchantia macropora, Musaceae, such as the genus Musa, for example the genera and species Musa nana, Musa acuminata, Musa paradisiaca, Musa spp. [banana], Onagraceae, such as the genera Camissonia, Oenothera, for example the genera and species Oenothera biennis or Camissonia brevipes [evening primrose], Palmae, such as the genus Elacis, for example the genus and species Elaeis guineensis [oil palm], Papaveraceae, such as the genus Papaver, for example the genera and species Papaver orientale, Papaver rhoeas, Papaver dubium [poppy], Pedaliaceae, such as the genus Sesamum, for example the genus and species Sesamum indicum [sesame], Piperaceae, such as the genera Piper, Artanthe, Peperomia, Steffensia, for example the genera and species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata [cayenne pepper], Poaceae, such as the genera Hordeum, Secale, Avena, Sorghum, Andropogon, Holcus, Panicum, Oryza, Zea (maize), Triticum, for example the genera and species Hordeum vulgare, Hordeum jubatum, Hordeum murinum, Hordeum secalinum, Hordeum distichon, Hordeum aegiceras, Hordeum hexastichon, Hordeum hexastichum, Hordeum irregulare, Hordeum sativum, Hordeum secalinum [barley], Secale cereale [rye], Avena sativa, Avena fatua, Avena byzantina, Avena fatua var. sativa, Avena hybrida [oats], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum, Panicum militaceum Oryza sativa, Oryza latifolia [rice], Zea mays [maize], Triticum aestivum, Triticum durum, Triticum turgidum, Triticum hybemum, Triticum macha, Triticum sativum or Triticum vulgare [wheat], Porphyridiaceae, such as the genera Chroothece, Flintiella, Petrovanella, Porphyridium, Rhodella, Rhodosorus, Vanhoeffenia, for example the genus and species Porphyridium cruentum, Proteaceae, such as the genus Macadamia, for example the genus and species Macadamia intergrifolia [macadamia], Prasinophyceae such as the genera Nephroselmis, Prasinococcus, Scherffelia, Tetraselmis, Mantoniella, Ostreococcus, for example the genera and species Nephroselmis olivacea, Prasinococcus capsulatus, Scherffelia dubia, Tetraselmis chui, Tetraselmis suecica, Mantoniella squamata, Ostreococcus tauri, Rubiaceae such as the genus Cofea, for example the genera and species Cofea spp., Coffea arabica, Coffea canephora or Coffea liberica [coffee], Scrophulariaceae such as the genus Verbascum, for example the genera and species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein], Solanaceae such as the genera Capsicum, Nicotiana, Solanum, Lycopersicon, for example the genera and species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [eggplant], Lycopersicon esculentum, Lycopersicon lycopersicum, Lycopersicon pyriforme, Solanum integrifolium or Solanum lycopersicum [tomato], Sterculiaceae, such as the genus Theobroma, for example the genus and species Theobroma cacao [cacao] or Theaceae, such as the genus Camellia, for example the genus and species Camellia sinensis [tea]. In particular preferred plants to be used as transgenic plants in accordance with the present invention are oil fruit crops which comprise large amounts of lipid compounds, such as peanut, oilseed rape, canola, sunflower, safflower, poppy, mustard, hemp, castor-oil plant, olive, sesame, Calendula, Punica, evening primrose, mullein, thistle, wild roses, hazelnut, almond, macadamia, avocado, bay, pumpkin/squash, linseed, soybean, pistachios, borage, trees (oil palm, coconut, walnut) or crops such as maize, wheat, rye, oats, triticale, rice, barley, cotton, cassava, pepper, Tagetes, Solanaceae plants such as potato, tobacco, eggplant and tomato, Vicia species, pea, alfalfa or bushy plants (coffee, cacao, tea), Salix species, and perennial grasses and fodder crops. Preferred plants according to the invention are oil crop plants such as peanut, oilseed rape, canola, sunflower, safflower, poppy, mustard, hemp, castor-oil plant, olive, Calendula, Punica, evening primrose, pumpkin/squash, linseed, soybean, borage, trees (oil palm, coconut). Especially preferred are plants which are high in C18:2- and/or C18:3-fatty acids, such as sunflower, safflower, tobacco, mullein, sesame, cotton, pumpkin/squash, poppy, evening primrose, walnut, linseed, hemp, thistle or safflower. Very especially preferred plants are plants such as safflower, sunflower, poppy, evening primrose, walnut, linseed, or hemp.

Preferred mosses are Physcomitrella or Ceratodon. Preferred algae are Isochrysis, Mantoniella, Ostreococcus or Crypthecodinium, and algae/diatoms such as Phaeodactylum or Thraustochytrium. More preferably, said algae or mosses are selected from the group consisting of: Shewanella, Physcomitrella, Thraustochytrium, Fusarium, Phytophthora, Ceratodon, Isochrysis, Aleurita, Muscarioides, Mortierella, Phaeodactylum, Cryphthecodinium, specifically from the genera and species Thallasiosira pseudonona, Euglena gracilis, Physcomitrella patens, Phytophtora infestans, Fusarium graminaeum, Cryptocodinium cohnii, Ceratodon purpureus, Isochrysis galbana, Aleurita farinosa, Thraustochytrium sp., Muscarioides viallii, Mortierella alpina, Phaeodactylum tricornutum or Caenorhabditis elegans or especially advantageously Phytophtora infestans, Thallasiosira pseudonona and Cryptocodinium cohnii.

Transgenic plants may be obtained by transformation techniques as published, and cited, in: Plant Molecular Biology and Biotechnology (CRC Press, Boca Raton, Fla.), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205-225. Preferably, transgenic plants can be obtained by T-DNA-mediated transformation. Such vector systems are, as a rule, characterized in that they contain at least the vir genes, which are required for the Agrobacterium-mediated transformation, and the sequences which delimit the T-DNA (T-DNA border). Suitable vectors are described elsewhere in the specification in detail.

Preferably, a multicellular micro-organism as used herein refers to protists or diatoms. More preferably, it is selected from the group of the families Dinophyceae, Turaniellidae or Oxytrichidae, such as the genera and species: Crypthecodinium cohnii, Phaeodactylum tricornutum, Stylonychia mytilus, Stylonychia pustulate, Stylonychia putrina, Stylonychia notophora, Stylonychia sp., Colpidium campylum or Colpidium sp.

The present invention also relates to a method for expressing a nucleic acid of interest in a host cell comprising
(a) introducing the polynucleotide or the vector of the present invention into the host cell; and
(b) expressing at least one nucleic acid of interest in said host cell.

The polynucleotide or vector of the present invention can be introduced into the host cell by suitable transfection or transformation techniques as specified elsewhere in this description. The nucleic acid of interest will be expressed in the host cell under suitable conditions. To this end, the host cell will be cultivated under conditions which, in principle, allow for transcription of nucleic acids. Moreover, the host cell, preferably, comprises the exogenously supplied or endogenously present transcription machinery required for expressing a nucleic acid of interest by the expression control sequence. More preferably, expressing in the method of the present invention refers to bidirectional expression of at least one nucleic acid of interest in each of the two orientations from the expression control sequence.

Moreover, the present invention encompasses a method for expressing a nucleic acid of interest in a non-human organism comprising
(a) introducing the polynucleotide or the vector of the present invention into the non human organism; and
(b) expressing at least one nucleic acid of interest in said non-human transgenic organism.

The polynucleotide or vector of the present invention can be introduced into the non-human transgenic organism by suitable techniques as specified elsewhere in this description. The non-human transgenic organism, preferably, comprises the exogenously supplied or endogenously present transcription machinery required for expressing a nucleic acid of interest by the expression control sequence. More preferably, expressing in the method of the present invention refers to bidirectional expression of at least one nucleic acid of interest in each of the two orientations from the expression control sequence.

In the following, some preferred embodiments pertaining to the present invention are described in more detail.

In a preferred embodiment, the polynucleotide of the present invention also comprises further genetic control sequences. A genetic control sequence as referred to in accordance with the present invention is to be understood broadly and means all sequences having an influence on the coming into existence of the function of the transgenic expression cassette of the invention. Genetic control sequences modify for example the transcription and translation in prokaryotic or eukaryotic organisms. The expression cassettes of the invention preferably comprise as additional genetic control sequence one of the promoters of the invention 5′-upstream from the particular nucleic acid sequence to be expressed transgenically, and a terminator sequence 3′-downstream, and if appropriate further usual regulatory elements, in each case functionally linked to the nucleic acid sequence to be expressed transgenically.

Genetic control sequences also comprise further promoters, promoter elements or minimal promoters which are able to modify the expression-controlling properties. It is thus possible for example through genetic control sequences for tissue-specific expression to take place additionally in dependence on particular stress factors. Corresponding elements are described for example for water stress, abscisic acid (Lam E and Chua N H, (1991) J Biol Chem 266(26):17131-17135) and heat stress (Schöffl F et al. (1989) Mol Gen Genetics 217(2-3):246-53). A further possibility is for further promoters which make expression possible in further plant tissues or in other organisms such as, for example, E. coli bacteria to be functionally linked to the nucleic acid sequence to be expressed. Suitable plant promoters are in principle all the promoters described above. It is conceivable for example that a particular nucleic acid sequence is described by a promoter (for example one of the promoters of the invention) in one plant tissue as sense RNA and translated into the corresponding protein, while the same nucleic acid sequence is transcribed by another promoter with a different specificity in a different tissue into antisense RNA, and the corresponding protein is down-regulated. This can be implemented by an expression cassette of the invention by the one promoter being positioned in front of the nucleic acid sequence to be expressed transgenically, and the other promoter behind.

Genetic control sequences further comprise also the 5′-untranslated region, introns or the noncoding 3′ region of genes, preferably of the pFD gene and/or of the OASTL gene. It has been shown that untranslated regions may play a significant functions in the regulation of gene expression. Thus, it has been shown that 5′-untranslated sequences may enhance the transient expression of heterologous genes. They may moreover promote tissue specificity (Rouster J et al. (1998) Plant J. 15:435-440.). Conversely, the 5′-untranslated region of the opaque-2 gene suppresses expression. Deletion of the corresponding region leads to an increase in gene activity (Lohmer S et al. (1993) Plant Cell 5:65-73). Further 5′-untranslated sequences and introns with expression-promoting function are known to the skilled worker. McElroy and coworkers (McElroy et al. (1991) Mol Gen Genet 231(1):150-160) reported on a construct based on the rice actin 1 (Act1) promoter for transforming monocotyledonous plants. Use of the Act1 intron in combination with the 35S promoter in transgenic rice cells led to an expression rate which was increased ten-fold compared with the isolated 35S promoter. Optimization of the sequence environment of the translation initiation site of the reporter gene gene (GUS) resulted in a four-fold increase in GUS expression in transformed rice cells. Combination of the optimized translation initiation site and of the Act1 intron resulted in a 40-fold increase in GUS expression by the CaMV35S promoter in transformed rice cells; similar results have been obtained with transformed corn cells. Overall, it was concluded from the investigations described above that the expression vectors based on the Act1 promoter are suitable for controlling sufficiently strong and constitutive expression of foreign DNA in transformed cells of monocotyledonous plants.

The expression cassette may comprise one or more so-called enhancer sequences functionally linked to the promoter, which make increased transgenic expression of the nucleic acid sequence possible. It is also possible to insert additional advantageous sequences, such as further regulatory elements or terminators, at the 3′ end of the nucleic acid sequences which are to be expressed transgenically.

Control sequences additionally mean those which make homologous recombination or insertion into the genome of a host organism possible or which allow deletion from the genome. It is possible in homologous recombination for example for the natural promoter of a particular gene to be replaced by one of the promoters of the invention. Methods such as the creaox technology permit tissue-specific deletion, which is inducible in some circumstances, of the expression cassette from the genome of the host organism (Sauer B. (1998) Methods. 14(4):381-92). In this case, particular flanking sequences are attached (lox sequences) to the target gene and subsequently make deletion possible by means of cre recombinase. The promoter to be introduced can be placed by means of homologous recombination in front of the target gene which is to be expressed transgenically by linking the promoter to DNA sequences which are, for example, homologous to endogenous sequences which precede the reading frame of the target gene. Such sequences are to be regarded as genetic control sequences. After a cell has been transformed with the appropriate DNA construct, the two homologous sequences can interact and thus place the promoter sequence at the desired site in front of the target gene, so that the promoter sequence is now functionally linked to the target gene and forms an expression cassette of the invention. The selection of the homologous sequences determines the promoter insertion site. It is possible in this case for the expression cassette to be generated by homologous recombination by means of single or double reciprocal recombination. In single reciprocal recombination there is use of only a single recombination sequence, and the complete introduced DNA is inserted. In double reciprocal recombination the DNA to be introduced is flanked by two homologous sequences, and the flanking region is inserted. The latter process is suitable for replacing, as described above, the natural promoter of a particular gene by one of the promoters of the invention and thus modifying the location and timing of gene expression. This functional linkage represents an expression cassette of the invention. To select successfully homologously recombined or else transformed cells it is usually necessary additionally to introduce a selectable marker. Various suitable markers are mentioned below. The selection marker permits selection of transformed from untransformed cells. Homologous recombination is a relatively rare event in higher eukaryotes, especially in plants. Random integrations into the host genome predominate. One possibility of deleting randomly integrated sequences and thus enriching cell clones having a correct homologous recombination consists of using a sequence-specific recombination system as described in U.S. Pat. No. 6,110,736.

Polyadenylation signals suitable as genetic control sequences are plant polyadenylation signals and-preferably-those from Agrobacterium tumefaciens.

In a particularly preferred embodiment, the expression cassette comprises a terminator sequence which is functional in plants. Terminator sequences which are functional in plants means in general sequences able to bring about termination of transcription of a DNA sequence in plants. Examples of suitable terminator sequences are the OCS (octopine synthase) terminator and the NOS (nopaline synthase) terminator. However, plant terminator sequences are particularly preferred. Plant terminator sequences means in general sequences which are a constituent of a natural plant gene. Particular preference is given in this connection to the terminator of the potato cathepsin D inhibitor gene (GenBank Acc. No.: X74985) or of the terminator of the field bean storage protein gene VfLEIB3 (GenBank Acc. No.: Z26489). These terminators are at least equivalent to the viral or T-DNA terminators described in the art.

The skilled worker is also aware of a large number of nucleic acids and proteins whose recombinant expression is advantageous under the control of the expression cassettes or processes of the invention. The skilled worker is further aware of a large number of genes through whose repression or switching off by means of expression of an appropriate antisense RNA it is possible likewise to achieve advantageous effects. Non-restrictive examples of advantageous effects which may be mentioned are: facilitated production of a transgenic organism for example through the expression of selection markers, achievement of resistance to abiotic stress factors (heat, cold, aridity, increased moisture, environmental toxins, UV radiation), achievement of resistance to biotic stress factors (pathogens, viruses, insects and diseases), improvement in human or animal food properties, improvement in the growth rate of the yield. Some specific examples of nucleic acids whose expression provides the desired advantageous effects may be mentioned below:

1. Selection Markers. Selection marker comprises both positive selection markers which confer resistance to an antibiotic, herbicide or biocide, and negative selection markers which confer sensitivity to precisely the latter, and markers which provide the transformed organism with a growth advantage (for example through expression of key genes of cytokine biosynthesis; Ebinuma H et al. (2000) Proc Natl Acad Sci USA 94:2117-2121). In the case of positive selection, only the organisms which express the corresponding selection marker thrive, whereas in the case of negative selection it is precisely these which perish. The use of a positive selection marker is preferred in the production of transgenic plants.

It is further preferred to use selection markers which confer growth advantages. Negative selection markers can be used advantageously if the intention is to delete particular genes or genome sections from an organism (for example as part of a crossbreeding process). The selectable marker introduced with the expression cassette confers resistance to a biocide (for example a herbicide such as phosphinothricin, glyphosate or bromoxynil), a metabolism inhibitor such as 2-deoxyglucose 6-phosphate (WO 98/45456) or an antibiotic such as, for example, kanamycin, G 418, bleomycin, hygromycin, on the successfully recombined or transformed cells. The selection marker permits selection of transformed from transformed from untransformed cells (McCormick et al. (1986) Plant Cell Rep 5:81-84). Particularly preferred selection markers are those which confer resistance to herbicides. The skilled worker is aware of numerous selection markers of this type and the sequences coding therefor. Non-restrictive examples may be mentioned below: i) Positive Selection Markers: The selectable marker introduced with the expression cassette confers resistance to a biocide (for example a herbicide such as phosphinothricin, glyphosate or bromoxynil), a metabolism inhibitor such as 2-deoxyglucose 6-phosphate (WO 98/45456) or an antibiotic such as, for example, tetracycline, ampicillin, kanamycin, G 418, neomycin, bleomycin or hygromycin, on the successfully transformed cells. The selection marker permits selection of transformed from untransformed cells (McCormick et al. (1986) Plant Cell Rep 5:81-84). Particularly preferred selection markers are those which confer resistance to herbicides. Examples of selection markers which may be mentioned are: DNA sequences which code for phosphinothricin acetyltransferases (PAT; also called bialophos resistance gene (bar)) and bring about detoxification of the herbicide phosphinothricin (PPT) (de Block et al. (1987) EMBO J 6:2513-2518). Suitable bar genes can be isolated from, for example, Streptomyces hygroscopicus or S. viridochromogenes. Corresponding sequences are known to the skilled worker (GenBank Acc. No.: X17220, X05822, M22827, X65195; U.S. Pat. No. 5,489,520). Also described are synthetic genes for example for expression in plastids AJ028212. A synthetic Pat gene is described in Becker et al. (1994) Plant J 5:299-307. The genes confer resistance to the herbicide bialaphos and are a widely used marker in transgenic plants (Vickers J E et al. (1996) Plant Mol Biol Rep 14:363-368; Thompson C J et al. (1987) EMBO J 6:2519-2523). 5-enolpyruvylshikimate-3-phosphate synthase genes (EPSP synthase genes) which confer resistance to glyphosate (N-(phosphonomethyl)glycine) (Steinrucken H C et al. (1980) Biochem Biophys Res Commun 94:1207-1212; Levin J G and Sprinson D B (1964) J Biol Chem 239:1142-1150; Cole D J (1985) Mode of action of glyphosate; A literature analysis, p. 48-74. In: Grossbard E and Atkinson D (eds.). The herbicide glyphosate. Buttersworths, Boston.). Glyphosate-tolerant EPSPS variants are preferably used as selection markers (Padgette S R et al. (1996). New weed control opportunities: development of soybeans with a Roundup Ready™ gene. In: Herbicide Resistant Crops (Duke S O ed.), pp. 53-84. CRC Press, Boca Raton, Fla.; Saroha M K and Malik V S (1998) J Plant Biochem Biotechnol 7:65-72). The EPSPS gene of the Agrobacterium sp. strain CP4 has a natural glyphosate tolerance which can be transferred to appropriate transgenic plants (Padgette S R et al. (1995) Crop Science 35(5):1451-1461). 5-Enolpyrvylshikimate-3-phosphate synthases which are glyphosate-tolerant are described for example in U.S. Pat. No. 5,510,471; U.S. Pat. No. 5,776,760; U.S. Pat. No. 5,864,425; U.S. Pat. No. 5,633,435; U.S. Pat. No. 5,627,061; U.S. Pat. No. 5,463,175; EP 0 218 571. Further sequences are described under GenBank Accession X63374. The aroA gene is further preferred (MI 0947). the gox gene (glyphosate oxide reductase from Achromobacter sp.) coding for the glyphosate-degrading enzymes. GOX can confer resistance to glyphosate (Padgette S R et al. (1996) J Nutr. 126(3):702-16; Shah D et al. (1986) Science 233: 478-481), the deh gene (coding for a dehalogenase which inactivates dalapon), (GenBank Acc. No.: AX022822, AX022820 and WO99/27116), bxn genes which code for bromoxynil-degrading nitrilase enzymes. For example the nitrilase from Klebsiella ozanenae. Sequences are to be found in GenBank for example under the Acc. No: E01313 and J03196. neomycin phosphotransferases confer resistance to antibiotics (aminoglycosides) such as neomycin, G418, hygromycin, paromomycin or kanamycin by reducing their inhibiting effect through a phosphorylation reaction. The nptll gene is particularly preferred. Sequences can be obtained from GenBank (AF080390 minitransposon mTn5-GNm; AF080389 minitransposon mTn5-Nm, complete sequence). In addition, the gene is already a component of numerous expression vectors and can be isolated therefrom by using processes familiar to the skilled worker (such as, for example, polymerase chain reaction) (AF234316 pCAMBIA-2301; AF234315 pCAMBIA-2300, AF234314 pCAMBIA-2201). The NPTII gene codes for an aminoglycoside 3′O-phosphotransferase from E. coli, Tn5 (GenBank Acc. No: U00004 Position 1401-2300; Beck et al. (1982) Gene 19 327-336), the DOG<R> 1 gene. The DOG<R> 1 gene was isolated from the yeast Saccharomyces cerevisiae (EP 0 807 836). It codes for a 2-deoxyglucose-6-phosphate phosphatase which confers resistance to 2-DOG (Randez-Gil et al. 1995, Yeast 11, 1233-1240; Sanz et al. (1994) Yeast 10:1195-1202, sequence: GenBank Acc. No.: NC001140 chromosome VIII, Saccharomyces cervisiae position 194799-194056). sulfonylureaand imidazolinone-inactivating acetolactate synthases which confer resistance to imidazolinone/sulfonylurea herbicides. Suitable examples are the sequence deposited under GenBank Acc No.: X51514 for the Arabidopsis thaliana Csr 1.2 gene (EC 4.1.3.18) (Sathasivan K et al. (1990) Nucleic Acids Res. 18(8):2188). Acetolactate synthases which confer resistance to imidazolinone herbicides are also described under GenBank Acc. No.: AB049823, AF094326, X07645, X07644, A19547, A19546, A19545, I05376, I05373, AL133315. hygromycin phosphotransferases (X74325 P. pseudomallei gene for hygromycin phosphotransferase) which confer resistance to the antibiotic hygromycin. The gene is a constituent of numerous expression vectors and can be isolated therefrom by using processes familiar to the skilled worker (such as, for example, polymerase chain reaction) (AF294981 pINDEX4; AF234301 pCAMBIA-1380; AF234300 pCAMBIA-1304; AF234299 pCAMBIA-1303; AF234298 pCAMBIA-1302; AF354046 pCAMBIA-1305; AF354045 pCAMBIA-1305.1) Resistance genes for a) chloramphenicol (chloramphenicol acetyltransferase), b) tetracycline, various resistance genes are described, e.g. X65876 S. ordonez genes class D teta and tetR for tetracycline resistance and repressor proteins X51366 Bacillus cereus plasmid pBC16 tetracycline resistance gene. In addition, the gene is already a constituent of numerous expression vectors and can be isolated therefrom by using processes familiar to the skilled worker (such as, for example, polymerase chain reaction) c) streptomycin, various resistance genes are described, e.g. with the GenBank Acc. No.: AJ278607 Corynebacterium acetoacidophilum ant gene for streptomycin adenylyltransferase. d) zeocin, the corresponding resistance gene is a constituent of numerous cloning vectors (e.g. L36849 cloning vector pZEO) and can be isolated therefrom by using processes familiar to the skilled worker (such as, for example, polymerase chain reaction). e) ampicillin ([beta]-lactamase gene; Datta N, Richmond M H. (1966) Biochem J. 98(1):204-9; Heffron F et al (1975) J. Bacteriol 122: 250-256; the Amp gene was first cloned to prepare the E. coli vector pBR322; Bolivar F et al. (1977) Gene 2:95-114). The sequence is a constituent of numerous cloning vectors and can be isolated therefrom by using processes familiar to the skilled worker (such as, for example, polymerase chain reaction). Genes such as the isopentenyltransferase from Agrobacterium tumefaciens (strain:PO22) (Genbank Acc. No.: AB025109). The ipt gene is a key enzyme in cytokine biosynthesis. Overexpression thereof facilitates regeneration of plants (e.g. selection on cytokine-free medium). The process for utilizing the ipt gene is described (Ebinuma H et al. (2000) Proc Natl Acad Sci USA 94:2117-2121; Ebinuma H et al. (2000) Selection of Marker-free transgenic plants using the onco-genes (ipt, rol A, B, C) of Agrobacterium as selectable markers, In Molecular Biology of Woody Plants. Kluwer Academic Publishers). Various further positive selection markers which confer a growth advantage on the transformed plants compared with untransformed ones, and processes for their use are described inter alia in EP-A 0 601 092. Examples which should be mentioned are [beta]-glucuronidase (in conjunction with, for example, cytokinin glucuronide), mannose-6-phosphate isomerase (in conjunction with mannose), UDP-galactose 4-epimerase (in conjunction with, for example, galactose), with particular preference for mannose-6-phosphate isomerase in conjunction with mannose. ii) Negative Selection Markers Negative selection markers make it possible for example to select organisms with successfully deleted sequences which comprise the marker gene (Koprek T et al. (1999) Plant J 19(6):719-726). In the case of negative selection, for example a compound which otherwise has no disadvantageous effect for the plant is converted into a compound having a disadvantageous effect by the negative selection marker introduced into the plant. Also suitable are genes which per se have a disadvantageous effect, such as, for example, thymidine kinase (TK), diphtheria toxin A fragment (DT-A), the codA gene product coding for a cytosine deaminase (Gleave A P et al. (1999) Plant Mol Biol. 40(2):223-35; Perera R J et al. (1993) Plant Mol. Biol 23(4): 793-799; Stougaard J (1993) Plant J 3:755-761), the cytochrome P450 gene (Koprek et al. (1999) Plant J 16:719-726), genes coding for a haloalkane dehalogenase (Naested H (1999) Plant J 18:571-576), the iaaH gene (Sundaresan V et al. (1995) Genes & Development 9:1797-1810) or the tms2 gene (Fedoroff N V & Smith D L (1993) Plant J 3:273-289).

The concentrations used in each case for the selection of antibiotics, herbicides, biocides or toxins must be adapted to the particular test conditions or organisms. Examples which may be mentioned for plants are kanamycin (Km) 50 mgA, hygromycin B 40 mg/l, phosphinothricin (ppt) 6 mgA. It is also possible to express functional analogs of said nucleic acids coding for selection markers. Functional analogs means in this connection all the sequences which have substantially the same function, i.e. are capable of selecting transformed organisms. It is moreover perfectly possible for the functional analog to differ in other features. It may for example have a higher or lower activity or else possess further functionalities.

2. Improved protection of the plant against abiotic stress factors such as aridity, heat, or cold for example through overexpression of antifreeze polypeptides from Myoxocephalus Scorpius (WO 00/00512), Myoxocephalus octodecemspinosus, the Arabidopsis thaliana transcription activator CBF1, glutamate dehydrogenases (WO 97/12983, WO 98/11240), calcium-dependent protein kinase genes (WO 98/26045), calcineurins (WO 99/05902), farnesyltransferases (WO 99/06580), Pei Z M et al., Science 1998, 282: 287-290), ferritin (Deak M et al., Nature Biotechnology 1999, 17:192-196), oxalate oxidase (WO 99/04013; Dunwell J M Biotechnology and Genetic Engeneering Reviews 1998, 15:1-32), DREB1A factor (dehydration response element B 1A; Kasuga Metal., Nature Biotechnology 1999, 17:276-286), genes of mannitol or trehalose synthesis such as trehalose-phosphate synthase or trehalose-phosphate phosphatase (WO 97/42326), or by inhibition of genes such as of trehalase (WO 97/50561). Particularly preferred nucleic acids are those coding for the transcriptional activator CBF1 from Arabidopsis thaliana (GenBank Acc. No.: U77378) of the antifreeze protein from Myoxocephalus octodecemspinosus (Gen Bank Acc. No.: AF306348) or functional equivalents thereof.

3. Expression of metabolic enzymes for use in the animal and human food sectors, for example expression of phytase and cellulases. Particular preference is given to nucleic acids such as the artificial cDNA coding for a microbial phytase (GenBank Acc. No.: A19451) or functional equivalents thereof.

4. Achievement of resistance for example to fungi, insects, nematodes and diseases through targeted secretion or accumulation of particular metabolites or proteins in the epidermis of the embryo. Examples which may be mentioned are glucosinolates (defense against herbivors), chitinases or glucanases and other enzymes which destroy the cell wall of parasites, ribosome-inactivating proteins (RIPs) and other proteins of the plants' resistance and stress responses, as are induced on injury or microbial attack of plants or chemically by, for example, salicylic acid, jasmonic acid or ethylene, lysozymes from non-plant sources such as, for example, T4 lysozyme or lysozyme from various mammals, insecticidal proteins such as Bacillus thuringiensis endotoxin, [alpha]-amylase inhibitor or protease inhibitors (cowpea trypsin inhibitor), glucanases, lectins such as phytohemagglutinin, wheatgerm agglutinin, RNAses or ribozymes. Particularly preferred nucleic acids are those coding for the chit42 endochitinase from Trichoderma harzianum (GenBank Acc. No.: S78423) or for the N-hydroxylating, multi-functional cytochrome P-450 (CYP79) proteins from Sorghum bicolor (GenBank Acc. No.: U32624) or functional equivalents thereof.

5. The accumulation of glucosinolates in plants of the Cardales genus, especially the oil seeds to protect from pests (Rask L et al. (2000) Plant Mol Biol 42:93-113; Menard R et al. (1999) Phytochemistry 52:29-35), expression of the Bacillus thuringiensis endotoxin under the control of the 35S CaMV promoter (Vaeck et al. (1987) Nature 328:33-37) or protection of tobacco against fungal attack by expression of a bean chitonase under the control of the CaMV promoter (Broglie et al. (1991) Science 254:1194-119, is known.

The expression of synthetic crylA(b) and crylA(c) genes which code for the lepidoptera-specific delta endotoxins from Bacillus thuringiensis can bring about resistance to insect pests in various plants. Thus, it is possible in rice to achieve resistance to two of the principal rice pests, the striped stem borer (Chilo suppressalis) and the yellow stem borer (Scirpophaga incertulas) (Cheng X et al. (1998) Proc Natl Acad Sci USA 95(6):2767-2772; Nayak P et al. (1997) Proc Natl Acad Sci USA 94(6):2111-2116).

6. Expression of genes which bring about accumulation of fine chemicals such as of tocopherols, tocotrienols or carotenoids. An example which may be mentioned is phytoene desaturase. Nucleic acids which code for the phytoene desaturase from Narcissus pseudonarcissus (GenBank Acc. No.: X78815) or functional equivalents thereof are preferred.

7. Production of neutraceuticals such as, for example, polyunsaturated fatty acids such as, for example, arachidonic acid or EP (eicosapentaenoic acid) or DHA (docosahexaenoic acid) by expression of fatty acid elongases and/or desaturases or production of proteins having an improved nutritional value such as, for example, having a high content of essential amino acids (e.g. the methionine-rich 2S albumin gene of the Brazil nut). Preferred nucleic acids are those which code for the methionine-rich 2S albumin from Bertholletia excelsa (GenBank Acc. No.: AB044391), the [Delta]6-acyllipid desaturase from Physcomitrella patens (GenBank Acc. No.: AJ222980; Girke et al. (1998) Plant J 15:3948), the [Delta]6-desaturase from Mortierelia alpina (Sakuradani et al. (1999) Gene 238:445-453), the [Delta]5-desaturase from Caenorhabditis elegans (Michaelson et al. 1998, FEBS Letters 439:215-218), the [Delta]5-fatty acid desaturase (des-5) from Caenorhabditis elegans (GenBank Acc. No.: AF078796), the [Delta]5-desaturase from Mortierella alpina (Michaelson et al. J Biol Chem 273:19055-19059), the [Delta]6-elongase from Caenorhabditis elegans (Beaudoin et al. (2000) Proc Natl. Acad Sci USA 97:6421-6426), the [Delta]6-elongase from Physcomitrella patens (Zank et al. (2000) Biochemical Society Transactions 28:654-657) or functional equivalents thereof.

8. Production of fine chemicals (such as, for example, enzymes) and pharmaceuticals (such as, for example, antibodies or vaccines as described in Hood E E, Jilka J M. (1999) Curr Opin Biotechnol. 10(4):382-6; Ma J K, Vine N D (1999) Curr Top Microbiol Immunol 236:275-92). It has been possible for example to produce recombinant avidin from chicken egg white and bacterial [beta]-glucuronidase (GUS) on a large scale in transgenic corn plants (Hood et al. (1999) Adv Exp Med Biol 464:127-47). These recombinant proteins from corn plants are marketed as high-purity biochemicals by Sigma Chemicals Co.

9. Achieving an increased storage ability in cells which normally comprise few storage proteins or lipids with the aim of increasing the yield of these substances, for example by expression of an acetyl-CoA carboxylase. Preferred nucleic acids are those which code for the acetyl-CoA carboxylase (accase) from Medicago sativa (GenBank Acc. No.: L25042) or functional equivalents thereof. Further examples of advantageous genes are mentioned for example in Dunwell J M (2000) J Exp Bot. 51 Spec No:487-96.

It is also possible to express functional analogs of said nucleic acids and proteins. Functional analogs means in this connection all the sequences which have substantially the same function, i.e. are capable of the function (for example a substrate conversion or signal transduction) like the protein mentioned by way of example too. It is moreover perfectly possible for the functional analog to differ in other features. It may for example have a higher or lower activity or else possess further functionalities. Functional analogs also means sequences which code for fusion proteins consisting of one of the preferred proteins and other proteins, for example a further preferred protein or else a signal peptide sequence.

Expression of the nucleic acids under the control of the promoters of the invention is possible in any desired cell compartment such as, for example, the endomembrane system, the vacuole and the chloroplasts. Desired glycosylation reactions, especially foldings and the like, are possible by utilizing the secretory pathway. Secretion of the target protein to the cell surface or secretion into the culture medium, for example on use of suspension-cultured cells or protoplasts, is also possible. The target sequences necessary for this purpose can thus be taken into account in individual vector variations and be introduced, together with the target gene to be cloned, into the vector through use of a suitable cloning strategy. It is possible to utilize as target sequences both gene-intrinsic, where present, or heterologous sequences. Additional heterologous sequences which are preferred for the functional linkage, but not restricted thereto, are further targeting sequences to ensure the subcellular localization in apoplasts, in the vacuole, in plastids, in the mitochondrion, in the endoplasmic reticulum (ER), in the cell nucleus, in elaioplasts or other compartments; and translation enhancers' such as the 5′ leader sequence from tobacco mosaic virus (Gallie et al. (1987) Nucl Acids Res 15 8693-8711) and the like. The process for transporting proteins which are not localized per se in the plastids in a targeted fashion into the plastids is described (Klosgen R B & Weil J H (1991) Mol Gen Genet 225(2):297-304; Van Breusegem F et al. (1998) Plant Mol Biol 38(3):491-496). Preferred sequences are

a) small subunit (SSU) of the ribulose-bisphosphate carboxylase (Rubisco ssu) from pea, corn, sunflower

b) transit peptides derived from genes of plant fatty acid biosynthesis such as the transit peptide of the plastidic acyl carrier protein (ACP), the stearyl-ACP desaturase, [beta]-ketoacyl-ACP synthase or the acyl-ACP thioesterase

c) the transit peptide for GBSSI (starch granule bound starch synthase 1)

d) LHCP II genes.

The target sequences may be linked to other target sequences which differ from the transit peptide-encoding sequences in order to ensure a subcellular localization in the apoplast, in the vacuole, in plastids, in the mitochondrion, in the endoplasmic reticulum (ER), in the cell nucleus, in elaioplasts or other compartments. It is also possible to employ translation enhancers such as the 5′ leader sequence from tobacco mosaic virus (Gallie et al. (1987) Nucl Acids Res 15:8693-8711) and the like.

The skilled worker is also aware that he need not express the genes described above directly by use of the nucleic acid sequences coding for these genes, or repress them for example by anti-sense. He can also use for example artificial transcription factors of the type of zinc finger proteins (Beerli R R et al. (2000) Proc Natl Acad Sci USA 97(4):1495-500). These factors bind in the regulatory regions of the endogenous genes which are to be expressed or repressed and result, depending on the design of the factor, in expression or repression of the endogenous gene. Thus, the desired effects can also be achieved by expression of an appropriate zinc finger transcription factor under the control of one of the promoters of the invention.

The expression cassettes of the invention can likewise be employed for suppressing or reducing replication or/and translation of target genes by gene silencing.

The expression cassettes of the invention can also be employed for expressing nucleic acids which mediate so-called antisense effects and are thus able for example to reduce the expression of a target protein.

Preferred genes and proteins whose suppression is the condition for an advantageous phenotype comprise by way of example, but non-restrictively:

a) polygalacturonase to prevent cell degradation and mushiness of plants and fruits, tomatoes for example. Preferably used for this purpose are nucleic acid sequences such as that of the tomato polygalacturonase gene (Gen Bank Acc. No.: X14074) or its homologs from other genera and species.

b) reduction in the expression of allergenic proteins as described for example in Tada Y et al. (1996) FEBS Lett 391(3):341-345 or Nakamura R (1996) Biosci Biotechnol Biochem 60(8):1215-1221.

c) changing the color of flowers by suppression of the expression of enzymes of anthocyan biosynthesis. Corresponding procedures are described (for example in Forkmann G, Martens S. (2001) Curr Opin Biotechnol 12(2):155-160). Preferably used for this purpose are nucleic acid sequences like that of flavonoid 3′-hydroxylase (GenBank Acc. No.: AB045593), of dihydroflavanol 4-reductase (GenBank Acc. No.: AF017451), of chalcone isomerase (GenBank Acc. No.: AF276302), of chalcone synthase (GenBank Acc. No.: AB061022), of flavanone 3-beta-hydroxylase (GenBank Acc. No.: X72592) or of flavone synthase II (GenBank Acc. No.: AB045592) or their homologs from other genera and species.

d) shifting the amylose/amylopectin content in starch by suppression of branching enzyme Q, which is responsible for [alpha]-1,6-glycosidic linkage. Corresponding procedures are described (for example in Schwall G P et al. (2000) Nat Biotechnol 18(5):551-554). Preferably used for this purpose are nucleic acid sequences like that of the starch branching enzyme II of potato (GenBank Acc. No.: AR123356; U.S. Pat. No. 6,169,226) or its homologs from other genera and species.

An “antisense” nucleic acid means primarily a nucleic acid sequence which is wholly or partly complementary to at least part of the sense strand of said target protein. The skilled worker is aware that he can use alternatively the cDNA or the corresponding gene as starting template for corresponding antisense constructs. The antisense nucleic acid is preferably complementary to the coding region of the target protein or a part thereof. The antisense nucleic acid may, however, also be complementary to the non-coding region of a part thereof. Starting from the sequence information for a target protein, an antisense nucleic acid can be designed in a manner familiar to the skilled worker by taking account of the base-pair rules of Watson and Crick. An antisense nucleic acid may be complementary to the whole or a part of the nucleic acid sequence of a target protein. In a preferred embodiment, the antisense nucleic acid is an oligonucleotide with a length of for example 15, 20, 25, 30, 35, 40, 45 or 50 nucleotides.

The antisense nucleic acid comprises in a preferred embodiment [alpha]-anomeric nucleic acid molecules. [alpha]-Anomeric nucleic acid molecules form in particular double-stranded hybrids with complementary RNA in which the strands run parallel to one another, in contrast to the normal [beta] units (Gaultier et al. (1987) Nucleic Acids Res 15:6625-6641). The use of the sequences described above in sense orientation is likewise encompassed and may, as is familiar to the skilled worker, lead to cosuppression. The expression of sense RNA to an endogenous gene may reduce or switch off its expression, similar to that described for antisense approaches (Goring et al. (1991) Proc Natl Acad Sci USA 88:1770-1774; Smith et al. (1990) Mol Gen Genet 224:447-481; Napoli et al. (1990) Plant Cell 2:279-289; Van der Krol et al. (1990) Plant Cell 2:291-299). It is moreover for the introduced construct to represent the gene to be reduced wholly or only in part. The possibility of translation is unnecessary.

It is also very particularly preferred to use processes such as gene regulation by means of double-stranded RNA (double-stranded RNA interference). Corresponding processes are known to the skilled worker and described in detail (e.g. Matzke M A et al. (2000) Plant Mol Biol 43:401-415; Fire A. et al (1998) Nature 391:806-811; WO 99/32619; WO 99/53050; WO 00/68374; WO 00/44914; WO 00/44895; WO 00/49035; WO 00/63364). Express reference is made to the processes and methods described in the indicated references. Highly efficient suppression of native genes is brought about here through simultaneous introduction of strand and complementary strand.

It is possible and advantageous to couple the antisense strategy with a ribozyme process. Ribozymes are catalytically active RNA sequences which, coupled to the antisense sequences, catalytically cleave the target sequences (Tanner N K. FEMS Microbiol Rev. 1999; 23 (3):257-75). This may increase the efficiency of an antisense strategy. Expression of ribozymes for reducing particular proteins is known to the skilled worker and described for example in EP-A1 0 291 533, EP-A1 0 321 201 and EP-A1 0 360 257. Suitable target sequences and ribozymes can be deteremined as described by Steinecke (Ribozymes, Methods in Cell Biology 50, Galbraith et al. eds. Academic Press, Inc. (1995), 449-460) by secondary structure calculations of ribozyme RNA and target RNA and by the interaction thereof (Bayley C C et al., Plant Mol Biol. 1992; 18(2):353-361; Lloyd A M and Davis R W et al., Mol Gen Genet. 1994 March; 242(6):653-657). Examples which should be mentioned are hammerhead ribozymes (Haselhoff and Gerlach (1988) Nature 334:585-591). Preferred ribozymes are based on derivatives of the tetrahymena L-19 IVS RNA (U.S. Pat. No. 4,987,071; U.S. Pat. No. 5,116,742). Further ribozymes having selectivity for an L119 mRNA can be selected (Bartel D and Szostak J W (1993) Science 261:1411-1418).

In a further embodiment, target protein expression can be reduced by using nucleic acid sequences which are complementary to regulatory elements of the target protein genes, form with the latter a triple helical structure and thus prevent gene transcription (Helene C (1991) Anticancer Drug Des. 6(6):569-84; Helene C et al. (1992) Ann NY Acad Sci 660:27-36; Maher L J (1992) Bioassays 14(12):807-815).

The bidirectional promoters of the invention are particularly advantageous when it is employed for regulating two enzymes of a metabolic pathway. 2′-Methyl-6-phytylhydroquinone methyltransferase and homogentisate phytyl-pyrophosphate-transferase, for example, can be expressed simultaneously via one of the bidirectional promoters of the invention, bringing about an increase in tocopherols. In addition, inhibition of homogentisate dioxygenase (for example by expression of a corresponding dsRNA) and overexpression of tyrosine aminotransferase leads to an increase in the tocopherol content. In carotenoid metabolism, inhibition of [alpha]-cyclase and overexpression of [beta]-cyclase leads to a change in the content of [alpha]-carotene and [beta]-carotene.

It is possible to prevent post-transcriptional silencing effects by parallel inhibition of the transcription of the SDE3 gene and overexpression of the recombinant protein (WO 02/063039).

Immunologically active parts of antibodies can also be advantageously expressed by using the promoters of the invention. Thus, for example, the heavy chain of an IgG1 antibody can be expressed in one direction, and the light chain in the other direction. The two form a functional antibody after translation (WO 02/101006).

A further possibility is to express simultaneously stress-related ion transporters (WO 03/057899) together with herbicide genes in order to increase the tolerance of environmental effects.

Many enzymes consist of two or more subunits, both of which are necessary for functioning. It is possible by means of one of the bidirectional promoters of the invention to express two subunits simultaneously. One example thereof is overexpression of the [alpha] and [beta] subunits of follicle stimulating human hormone.

A construct consisting of a gene for a selection marker and a reporter gene is particularly valuable for establishing transformation systems, when they are regulated by this bidirectional promoter.

The expression cassettes of the invention and the vectors derived therefrom may comprise further functional elements. The term functional element is to be understood broadly and means all elements which have an influence on production, multiplication or function of the expression cassettes of the invention or vectors or organisms derived therefrom. Non-restrictive examples which may be mentioned are:

a) Reporter genes or proteins code for easily quantifiable proteins and ensure via an intrinsic color or enzymic activity an assessment of transformation efficiency or of the site or time of expression (Schenborn E, Groskreutz D (1999) Mol Biotechnol 13(1):2944). Examples which should be mentioned are: green fluorescence protein (GFP) (Chuff W L et al., Curr Biol 1996, 6:325-330; Leffel S M et al., Biotechniques. 23(5):912-8, 1997; Sheen et al. (1995) Plant Journal 8(5):777-784; Haseloff et al. (1997) Proc Natl Acad Sci USA 94(6):2122-2127; Reichel et al. (1996) Proc Natl Acad Sci USA 93(12):5888-5893; Tian et al. (1997) Plant Cell Rep 16:267-271; WO 97/41228), chloramphenicol transferase (Fromm et al. (1985) Proc Natl Acad Sci USA 82:5824-5828), luciferase (Millar et al. (1992) Plant Mol Biol Rep 10:324-414; Ow et al. (1986) Science, 234:856-859); permits detection of bioluminescence., [beta]-galactosidase, codes for an enzyme for which various chromogenic substrates are available, [beta]-glucuronidase (GUS) (Jefferson et al. (1987) EMBO J 6:3901-3907) or the uidA gene which encodes an enzyme for various chromogenic substrates, R-locus gene product protein which regulates the production of anthocyanin pigments (red coloration) in plant tissues and thus makes direct analysis possible of the promoter activity without adding additional auxiliaries or chromogenic substrates (Dellaporta et al., In: Chromosome Structure and Function: Impact of New Concepts, 18th Stadler Genetics Symposium 11:263-282, 1988), [beta]-lactamase (Sutcliffe (1978) Proc Natl Acad Sci USA 75:3737-3741), enzyme for various chromogenic substrates (e.g. PADAC, a chromogenic cephalosporin), xylE gene product (Zukowsky et al. (1983) Proc Natl Acad Sci USA 80:1101-1105), catechol dioxygenase, which can convert chromogenic catechols, alpha-amylase (Ikuta et al. (1990) Biol Technol. 8:241-242, tyrosinase (Katz et al. (1983) J Gen Microbiol 129:2703-2714), enzyme which oxidizes tyrosine to DOPA and dopaquinone which subsequently form the easily detectable melanin, aequorin (Prasher et al. (1985) Biochem Biophys Res Commun 126(3):1259-1268), can be used in calcium-sensitive bioluminescence detection.

b) Origins of replication which ensure a multiplication of the expression cassettes or vectors of the invention in, for example, E. coli. Examples which may be mentioned are ORI (origin of DNA replication), the pBR322 ori or the P15A ori (Sambrook et al.: Molecular Cloning. A Laboratory Manual, 2nd ed. Cold Spring Harbor Laboratory Press, Cold Spring Harbor, N.Y., 1989).

c) Elements for example “border sequences” which make agrobacteria-mediated transfer into plant cells possible for transfer and integration into the plant genome, such as, for example, the right or left border of the T-DNA or the vir region.

d) Multiple cloning regions (MCS) permit and facilitate the insertion of one or more nucleic acid sequences.

The skilled worker is aware of various ways of obtaining an expression cassette of the invention. The production of an expression cassette of the invention takes place for example by fusing one of the expression control sequence of the invention with a nucleic acid sequence of interest to be expressed, if appropriate with a sequence coding for a transit peptide, preferably a chloroplast-specific transit peptide which is preferably disposed between the promoter and the respective nucleic acid sequence, and with a terminator or polyadenylation signal. Conventional techniques of recombination and cloning are used for this purpose (as described above).

However, and expression cassette also means constructions in which the promoter, without previously having been functionally linked to a nucleic acid sequence to be expressed, is introduced into a host genome, for example via a targeted homologous recombination or a random insertion, there assumes regulatory control of nucleic acid sequences which are then functionally linked to it, and controls transgenic expression thereof. Insertion of the promoter-for example by homologous recombination-in front of a nucleic acid coding for a particular polypeptide results in an expression cassette of the invention which controls the expression of the particular polypeptide in the plant. The insertion of the promoter may also take place by expression of antisense RNA to the nucleic acid coding for a particular polypeptide. Expression of the particular polypeptide in plants is thus downregulated or switched off.

It is also possible analogously for a nucleic acid sequence to be expressed transgenically to be placed, for example by homologous recombination, behind the endogenous, natural promoter, resulting in an expression cassette of the invention which controls the expression of the nucleic acid sequence to be expressed transgenically.

In principle, the invention also contemplates cells, cell cultures, parts-such as, for example, roots, leaves etc. in the case of transgenic plant organisms and transgenic propagation material such as seeds or fruits, derived from the transgenic organisms described above.

Genetically modified plants of the invention which can be consumed by humans and animals may also be used as human food or animal food for example directly or after processing in a manner known per se.

A further aspect of the invention, thus, relates to the use of the transgenic organisms of the invention described above and of the cells, cell cultures, parts-such as, for example, roots, leaves etc. in the case of transgenic plant organisms-and transgenic propagation material such as seeds or fruits derived therefrom for producing human or animal foods, pharmaceuticals or fine chemicals.

Preference is further given to a process for the recombinant production of pharmaceuticals or fine chemicals in host organisms, where a host organism is transformed with one of the expression cassettes or vectors described above, and this expression cassette comprises one or more structural genes which code for the desired fine chemical or catalyze the biosynthesis of the desired fine chemical, the transformed host organism is cultured, and the desired fine chemical is isolated from the culture medium. This process is widely applicable to fine chemicals such as enzymes, vitamins, amino acids, sugars, fatty acids, natural and synthetic flavorings, aromatizing substances and colorants. The production of tocopherols and tocotrienols, and of carotenoids is particularly preferred. The culturing of the transformed host organisms, and the isolation from the host organisms or from the culture medium takes place by means of processes known to the skilled worker. The production of pharmaceuticals such as, for example, antibodies or vaccines is described in Hood E E, Jilka J M (1999). Curr Opin Biotechnol 10(4):382-6; Ma J K, Vine N D (1999). Curr Top Microbiol Immunol 236:275-92.

All references cited in this specification are herewith incorporated by reference with respect to their entire disclosure content and the disclosure content specifically mentioned in this specification.

### FIGURES

FIG. 1: Sequences of the TaAffx.115437.1.A1 (SEQ ID NO: 6) and the maize ortholog Zm.348.2.A1_a_at (SEQ ID NO: 7).

FIG. 2: Zm.348.2.A1_a_at expression profiles using the Affymetrix maize chip hybridization. Tissues: 1-6: immature embryo; 7-14: leaf; 15-25: young ear; and 26-36: kernel.

FIG. 3: Sequence of the maize EST ZM03MC02483_{—}60578324 (SEQ ID NO: 8).

FIG. 4: qRT-PCR results of the ZM03MC02483_{—}60578324.

FIG. 5: (A) The corresponding CDS sequence of the ZmNP27 (SEQ ID NO: 4) and (B) the predicted protein (SEQ ID NO: 5).

FIG. 6: The sequence of ZmGSStuc11-12-04.271010.1 containing the predicted promoter region and partial corresponding coding sequence (SEQ ID No: 9).

FIG. 7: Sequence of Promoter ZmNP27 (pZmNP27; SEQ ID NO: 1).

FIG. 8: A binary Vector containing GUS expression cassette driven by the ZmNP27 promoter (RLN 88).

FIG. 9: Sequence of RLN 88 (SEQ ID NO: 10).

FIG. 10: The expression cassette of both GUS and DsRed reporter genes driven by the ZmNP27 promoter in bi-directions in the construct, RHF 175.

FIG. 11: Sequence of vector RHF175 (SEQ ID NO: 11).

FIG. 12: GUS expression in different tissues at different developmental stages driven by ZmNP27 in forward direction in transgenic maize with RLN88.

FIG. 13: Bi-directional function of the pZmNP27. The expression of DsRed gene was controlled by the pZmNP27 in reverse direction. The expression of GUS expression was controlled by pZmNP27 in forward direction in transgenic maize with RHF175.

FIG. 14: Sequence of pZmNP18 (SEQ ID NO: 2).

FIG. 15: Sequence of pZmNP27-mini (SEQ ID NO: 3).

FIG. 16: GUS expression in different tissues at different developmental stages driven by pZmNP18 in transgenic maize with RLN87.

FIG. 17: GUS expression in different tissues at different developmental stages driven by pZmNP27-mini in transgenic maize with RHF178.

### EXAMPLES

The invention will now be illustrated by the following Examples which are not intended, whatsoever, to limit the scope of this application.

### Example 1

### Identification of the Maize Ortholog of NP27

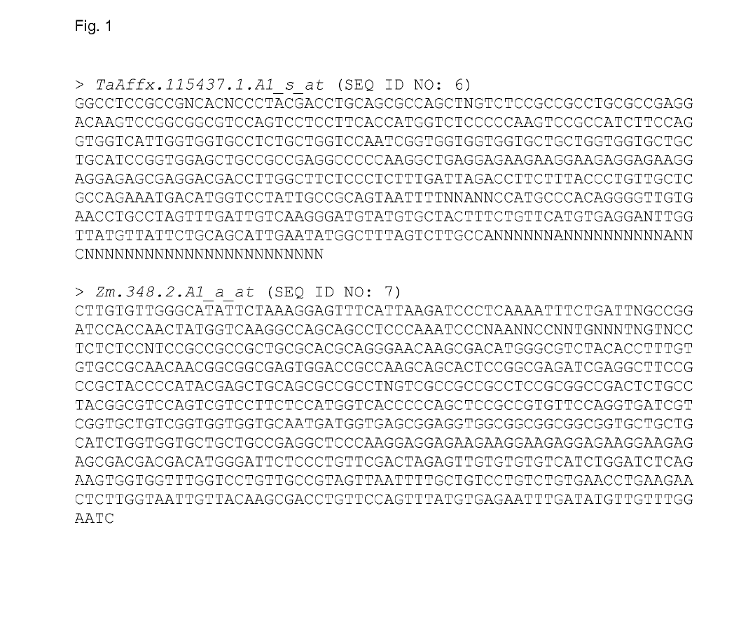

In an expression profiling analysis using Affymetrix GeneChip® Wheat Genome Arrays, the wheat chip consensus sequence TaAffx.115437.1.A1 showed constitutive expression. When the sequence of TaAffx.115437.1.A1 was aligned with the sequences of the Affymetrix maize chip, a maize chip consensus sequence, Zm.348.2.A1_a_at was identified as an ortholog of TaAffx.115437.1.A1 with 78% nucleotide sequence identity in the first 290 nucleotides of the TaAffx.115437.1.A1. The sequences of TaAffx.115437.1.A1 and Zm.348.2.A1_a_at are shown in FIG. 1.

### Example 2

### The Expression Profiles of Zm.348.2.A1_a_at Using Affymetrix GeneChip® Maize Genome Array Analysis

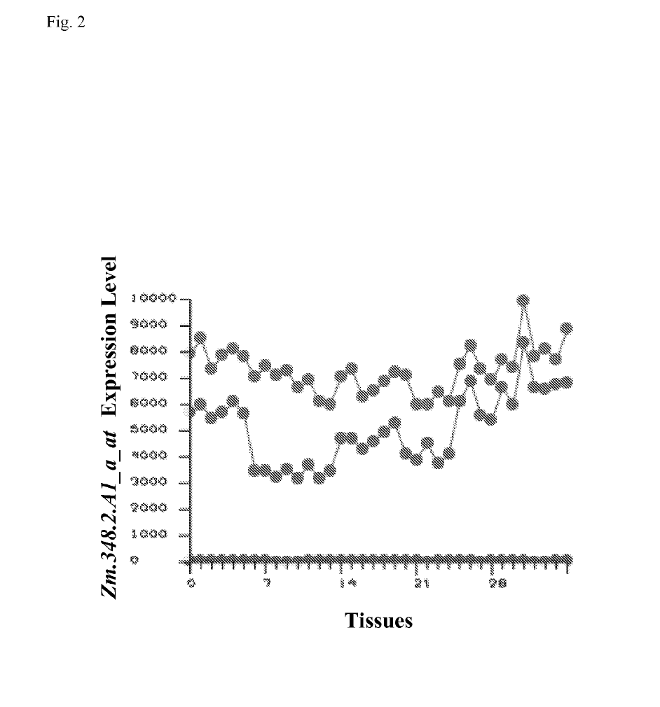

Total RNA isolated from immature embryo, leaf, young ear, and kernel was used for this Affymetrix GeneChip® Maize Genome Array analysis. A total of 36 arrays were hybridized. The results indicated that Zm.348.2.A1_a_at expressed constitutively in all tested tissues (FIG. 2).

### Example 3

### Validation of the Expression Profiling Data of Zm.348.2.A1_a_at Using Quantitative Reverse Transcriptase-polymerase Chain Reaction (qRT-PCR)

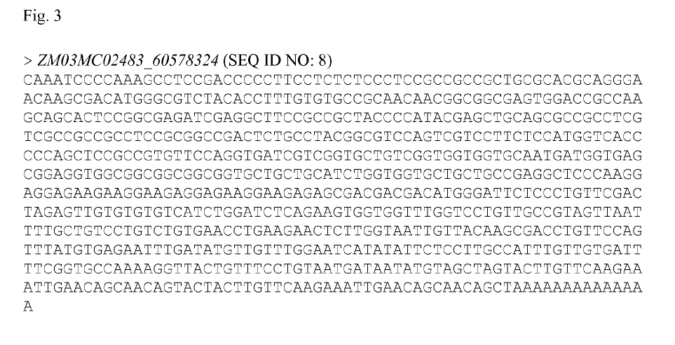

Quantitative reverse transcriptase-polymerase chain reaction (qRT-PCR) was performed to determine the expression levels of Zm.348.2.A1_a_at in various types of tissues. The sequence of Zm.348.2.A1_a_at was Blasted against the BASF Plant Science proprietary sequence database. One maize EST ZM03MC02483_{—}60578324 (745 bp) was identified as a member of the gene family of Zm.348.2.A1_a_at. The sequence of ZM03MC02483_{—}60578324 is shown in FIG. 3.

Primers for qRT-PCR were designed based on the sequence of ZM03MC02483_{—}60578324 using VNTI. Two sets of primers were used for PCR amplification. The sequences of primers are in Table 1. The glyceraldehyde-3-phosphate dehydrogenase (GAPDH) gene served as a control for normalization.

TABLE 1
Primer sequences for RT-QPCR

| Primer | Sequence (SEQ ID NO) |
|---|---|
| ZM03MC02483_60578324_Forward_1 | AACAAGCGACATGGGCGTCTA (12) |
| ZM03MC02483_60578324_Reverse_1 | AAGGACGACTGGACGCCGTA (13) |
| ZM03MC02483_60578324_Forward_2 | CGACATGGGCGTCTACACCTT (14) |
| ZM03MC02483_60578324_Reverse_2 | AAGGACGACTGGACGCCGTA (15) |
| GAPDH_Forward | GTAAAGTTCTTCCTGATCTGAAT (16) |
| GAPDH_Reverse | TCGGAAGCAGCCTTAATA (17) |

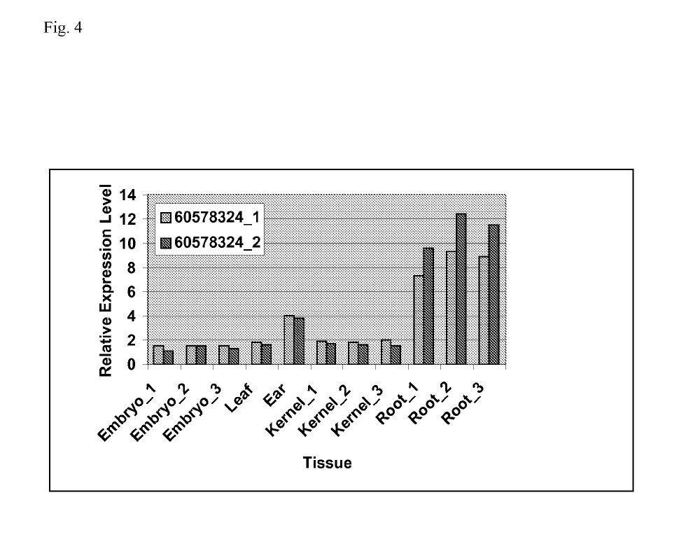

qRT-PCR was performed using SuperScript III Reverse Transcriptase (Invitrogen, Carlsbad, Calif., USA) and SYBR Green QPCR Master Mix (Eurogentec, San Diego, Calif., USA) in an ABI Prism 7000 sequence detection system. cDNA was synthesized using 2-3 □g of total RNA and 1 μL reverse transcriptase in a 20 □L volume. The cDNA was diluted to a range of concentrations (15-20 ng/□L). Thirty to forty ng of cDNA was used for quantitative PCR (qPCR) in a 30 □L volume with SYBR Green QPCR Master Mix following the manufacturer's instruction. The thermocycling conditions were as follows: incubate at 50° C. for 2 minutes, denature at 95° C. for 10 minutes, and run 40 cycles at 95° C. for 15 seconds and 60° C. for 1 minute for amplification. After the final cycle of the amplification, the dissociation curve analysis was carried out to verify that the amplification occurred specifically and no primer dimer was produced during the amplification process. The housekeeping gene glyceraldehyde-3-phosphate-dehydrogenase (GAPDH, primer sequences in Table 1) was used as an endogenous reference gene to normalize the calculation using Comparative Ct (Cycle of threshold) value method. The ΔC_{T} value was obtained by subtracting the Ct value of GAPDH gene from the Ct value of the candidate gene (ZM03MC02483_{—}60578324). The relative transcription quantity (expression level) of the candidate gene was given by 2^{-ΔCT}. The qRT-PCR results were summarized in FIG. 4. Both primer sets gave the similar expression patterns that are validated to the expression patterns obtained from the Affymetrix GeneChip® Maize Genome Array analysis shown in FIG. 2.

### Example 4

### Annotation of the Zm.348.2.A1_a_at Sequence

The coding sequence corresponding to the Zm.348.2.A1_a_at gene was annotated based on the in silico results obtained from both BlastX of EST ZM03MC02483_{—}60578324 sequence against GenBank protein database (nr) and result from VNTI translation program. The EST ZM03MC02483_{—}60578324 encodes a 60S acidic ribosomal protein P3 (GenBank Accession: O24413/RLA3_Maize) gene in maize. The top 15 homologous of the BlastX results are presented in table 2.

TABLE 2
BlastX search results of the maize EST ZM03MC02483_60578324

| Accession | Description | Score | E-value |
|---|---|---|---|
| O24413 | maize 60S acidic ribosomal protein P3 (P1/P2-like) (P3A). | 236 | 3.00E−61 |
| NP_001058484.1 | Os06g0701400 [Oryza sativa (japonica) | 192 | 6.00E−48 |
| NP_001042497.1 | Os01g0231700 [Oryza sativa (japonica | 189 | 4.00E−47 |
| NP_200539.1 | structural constituent of ribosome (Arabidopsis thaliana) | 146 | 4.00E−34 |
| NP_194319.1 | structural constituent of ribosome (Arabidopsis thaliana) | 145 | 5.00E−34 |
| AAR22555.1 | 60S acidic ribosomal protein P3 (Lactuca sativa) | 129 | 4.00E−29 |
| AAR22560.1 | 60S acidic ribosomal protein P3 (Lactuca sativa) | 128 | 8.00E−29 |
| AAR22559.1 | 60S acidic ribosomal protein P3 (Lactuca sativa) | 128 | 8.00E−29 |
| AAR22558.1 | 60S acidic ribosomal protein P3 (Lactuca sativa) | 122 | 7.00E−27 |
| AAR22557.1 | 60S acidic ribosomal protein P3 (Lactuca sativa) | 108 | 7.00E−23 |
| XP_001221957.1 | hypothetical protein CHGG_05862 [Chaetomium globosum CBS 148.51] | 74 | 3.00E−12 |
| XP_360515.1 | hypothetical protein MG10827.4 [Magnaporthe grisea 70-15] | 71 | 2.00E−11 |
| ABA40434.1 | 60s acidic ribosomal protein-like protein [Solanum tuberosum] | 70 | 4.00E−11 |
| EAT88488.1 | hypothetical protein SNOG_04728 [Phaeosphaeria nodorum SN15] | 68 | 1.00E−10 |
| EAU80487.1 | hypothetical protein CC1G_11842 [Coprinopsis cinerea okayama7^{••} 130] | 67 | 2.00E−10 |

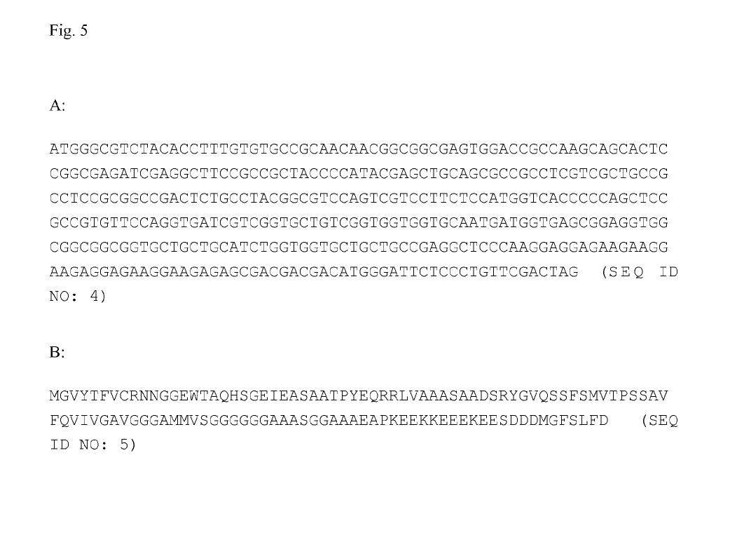

Accession: The CDS sequence identified using VNTI (GenBank O24413/RLA3_Maize, maize 60S acidic ribosomal protein P3 gene) was shown in FIG. 5 (A) and the translated amino acid sequence is shown in FIG. 5 (B).

### Example 5

### Identification of the Promoter Region

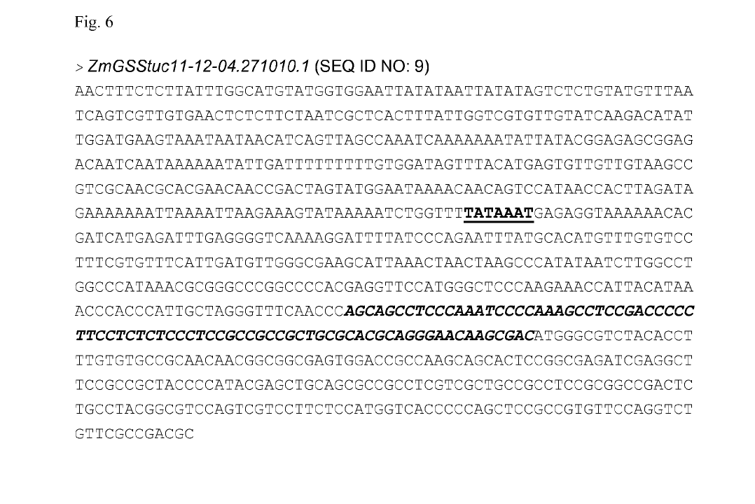

Sequence upstream of the start codon of the 60S acidic ribosomal protein P3 gene was defined as the promoter. To identify this predicted promoter region, the sequence of EST ZM03MC02483_{—}60578324 was mapped to the BASF Plant Science proprietary genomic DNA sequence database. One maize genomic DNA sequence, ZmGSStuc11-12-04.271010.1 (880 bp) was identified. This 880 bp sequence harboured a part of the EST ZM03MC02483_{—}60578324 and contained partial coding sequence (CDS) of the gene and 666 bp sequence upstream of the start codon (FIG. 6). The 5′ UTR (81 bp) was determined by the 5′RACE (Rapid Amplification of 5′ Complementary DNA Ends) and is indicated in bold and italic letters in FIG. 6. The putative TATA signal sequence is indicated in underlined bold letters (FIG. 6).

### Example 6

### Isolation of the Promoter Region by PCR Amplification

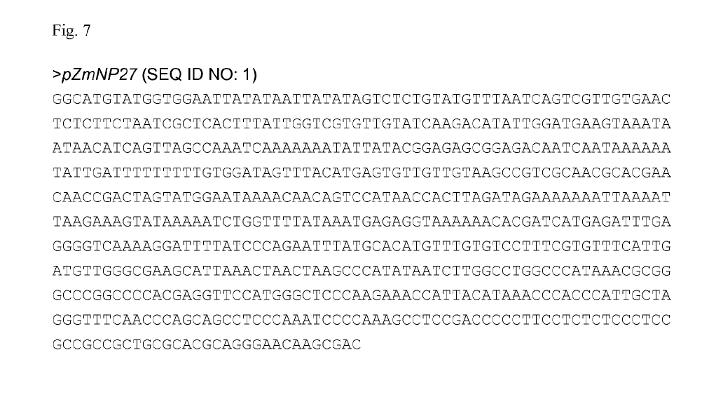

PCR was carried out using the sequence specific forward primer GGCATGTATGGTGGAATTAT (SEQ ID NO: 18) and reverse primer GTCGCTTGTTCCCTGCGTGC (SEQ ID NO: 19) to isolate the promoter region. A fragment of 651 bp was amplified from maize genomic DNA. This promoter region was named promoter ZmNP27 (pZmNP27). Sequence of pZmNP27 was shown in FIG. 7.

### Example 7

### PLACE Analysis and Prediction of Bi-directional Function of the Promoter ZmNP27

Cis-acting motifs in the 651 by ZmNP27 promoter region were identified using PLACE (a database of Plant Cis-acting Regulatory DNA elements) via Genomatix. The results were listed in Table 3. A putative TATA box is located between the nucleotide (nt) sequence number 335 and 341 in the forward strand. Two putative TATA boxes are located between the nucleotide (nt) sequence number 17 and 23 as well as 25 and 32 in the reverse strand and two CCAAT boxes are located between the nucleotide (nt) sequence number 84 and 88 as well as 108 and 112 in the reverse strand. The results of this in silico analysis indicated that the pZmNP27 might function as a bi-directional promoter.

TABLE 3
PLACE analysis results of the 651 bp ZmNP27 promoter

| | Start | End | | | | |
|---|---|---|---|---|---|---|
| IUPAC (SEQ ID NO) | pos. | pos. | Strand | Mismatches | Score | Sequence |
| POLLEN2LELAT52 (20) | 7 | 15 | − | 0 | 1 | TCCACCATA |
| TATABOX4 (21) | 17 | 23 | − | 0 | 1 | TATATAA |
| TATABOX4 (22) | 18 | 24 | + | 0 | 1 | TATATAA |
| TATABOX4 (23) | 25 | 31 | − | 0 | 1 | TATATAA |
| SURECOREATSULTR11 | 30 | 36 | − | 0 | 1 | GAGACTA |
| (24) | | | | | | |
| OSE2ROOTNODULE (25) | 64 | 68 | + | 0 | 1 | CTCTT |
| NTBBFIARROLB (26) | 79 | 85 | + | 0 | 1 | ACTTTAT |
| TAAAGSTKST1 (27) | 80 | 86 | − | 0 | 1 | AATAAAG |
| CCAATBOX1 (28) | 84 | 88 | − | 0 | 1 | CCAAT |
| RAV1AAT (29) | 92 | 96 | − | 0 | 1 | CAACA |
| CCAATBOX1 (30) | 108 | 112 | − | 0 | 1 | CCAAT |
| MYB2AT (31) | 131 | 141 | − | 0 | 1 | TGGCTAACTGA |
| ARFAT (32) | 169 | 175 | − | 0 | 1 | TTGTCTC |
| SURECOREATSULTR11 | 169 | 175 | + | 0 | 1 | GAGACAA |
| (33) | | | | | | |
| SEBFCONSSTPR10A | 169 | 175 | − | 0 | 1 | TTGTCTC |
| (34) | | | | | | |
| TATCCAYMOTIFOSRA- | 202 | 208 | − | 0 | 1 | TATCCAC |
| MY (35) | | | | | | |
| SV40COREENHAN (36) | 202 | 209 | + | 0 | 1 | GTGGATAG |
| TATCCACHVAL21 (37) | 202 | 208 | − | 0 | 1 | TATCCAC |
| MYBST1 (38) | 203 | 209 | + | 0 | 1 | TGGATAG |
| PREATPRODH (39) | 215 | 220 | − | 0 | 1 | ACTCAT |
| RAV1AAT (40) | 220 | 224 | − | 0 | 1 | CAACA |
| RAV1AAT (41) | 223 | 227 | − | 0 | 1 | CAACA |
| HEXAMERATH4 (42) | 232 | 237 | + | 0 | 1 | CCGTCG |
| CGACGOSAMY3 (43) | 233 | 237 | − | 0 | 1 | CGACG |
| DRE2COREZMRAB17 | 251 | 257 | + | 0 | 1 | ACCGACT |
| (44) | | | | | | |
| RAV1AAT (45) | 272 | 276 | + | 0 | 1 | CAACA |
| MYB1AT (46) | 282 | 287 | + | 0 | 1 | TAACCA |
| BOXIINTPATPB (47) | 293 | 298 | + | 0 | 1 | ATAGAA |
| GT1GMSCAM4 (48) | 296 | 301 | + | 0 | 1 | GAAAAA |
| CCA1ATLHCB1 (49) | 322 | 329 | + | 0 | 1 | AAAAATCT |
| MYB1AT (50) | 329 | 334 | − | 0 | 1 | AAACCA |
| TATABOX2 (51) | 335 | 341 | + | 0 | 1 | TATAAAT |
| ELRECOREPCRP1 (52) | 367 | 381 | − | 0 | 1 | TTTGACCCCT- |
| | | | | | | CAAAT |
| PYRIMIDINEBOXOSRAM | 379 | 384 | − | 0 | 1 | CCTTTT |
| (53) | | | | | | |
| IBOXCORE (54) | 386 | 392 | − | 0 | 1 | GATAAAA |
| SREATMSD (55) | 387 | 393 | + | 0 | 1 | TTTATCC |
| MYBST1 (56) | 388 | 394 | − | 0 | 1 | GGGATAA |
| MYCATERD (57) | 404 | 410 | − | 0 | 1 | CATGTGC |
| MYCATRD2 (58) | 405 | 411 | + | 0 | 1 | CACATGT |
| RAV1AAT (59) | 436 | 440 | − | 0 | 1 | CAACA |
| MYB1LEPR (60) | 453 | 459 | − | 0 | 1 | GTTAGTT |
| SITEIIATCYTC (61) | 457 | 467 | − | 0 | 1 | TGGGCTTAGTT |
| SITEIIATCYTC (62) | 477 | 487 | − | 0 | 1 | TGGGCCAGGC |
| | | | | | | C |
| UP1ATMSD (63) | 481 | 491 | + | 0 | 1 | TGGCCCATAAA |
| SORLIP2AT (64) | 491 | 501 | − | 0 | 1 | CGGGCCCGCG |
| | | | | | | T |
| CGCGBOXAT (65) | 491 | 496 | + | 0 | 1 | ACGCGG |
| CGCGBOXAT (66) | 491 | 496 | − | 0 | 1 | CCGCGT |
| SORLIP2AT (67) | 494 | 504 | + | 0 | 1 | CGGGCCCGGC |
| | | | | | | C |
| SORLIP2AT (68) | 496 | 506 | − | 0 | 1 | GGGGCCGGGC |
| | | | | | | C |
| SITEIIATCYTC (69) | 518 | 528 | + | 0 | 1 | TGGGCTCCCAA |
| MYB1AT (70) | 530 | 535 | + | 0 | 1 | AAACCA |
| SEF3MOTIFGM (71) | 543 | 548 | + | 0 | 1 | AACCCA |
| UP2ATMSD (72) | 556 | 564 | − | 0 | 1 | AAACCCTAG |
| SEF3MOTIFGM (73) | 566 | 571 | + | 0 | 1 | AACCCA |
| ANAERO2CONSENSUS | 571 | 576 | + | 0 | 1 | AGCAGC |
| (74) | | | | | | |
| LTRECOREATCOR15 | 596 | 602 | + | 0 | 1 | TCCGACC |
| (75) | | | | | | |
| GCCCORE (76) | 620 | 626 | + | 0 | 1 | CGCCGCC |

### Example 8

### Binary Vector Construction for Maize Transformation to Identify the Function of pZmNP27 in Forward Direction

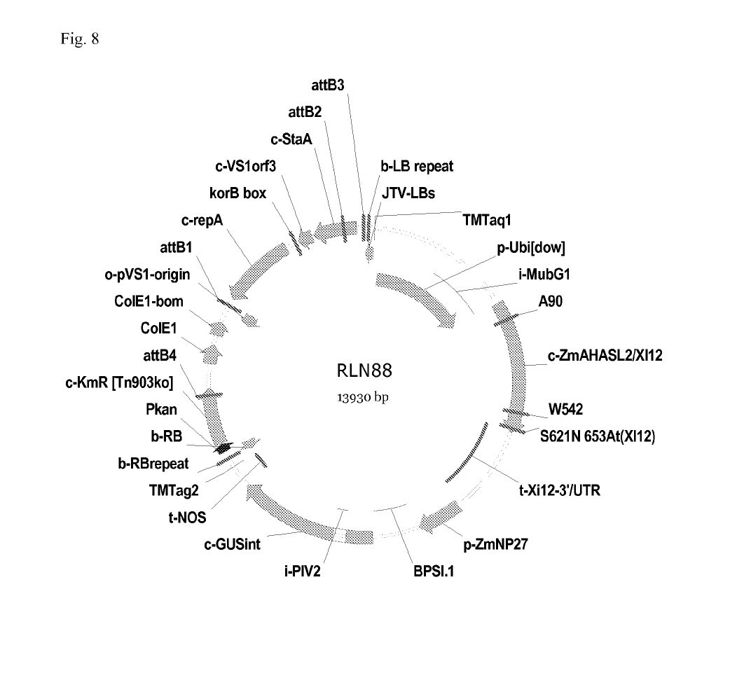

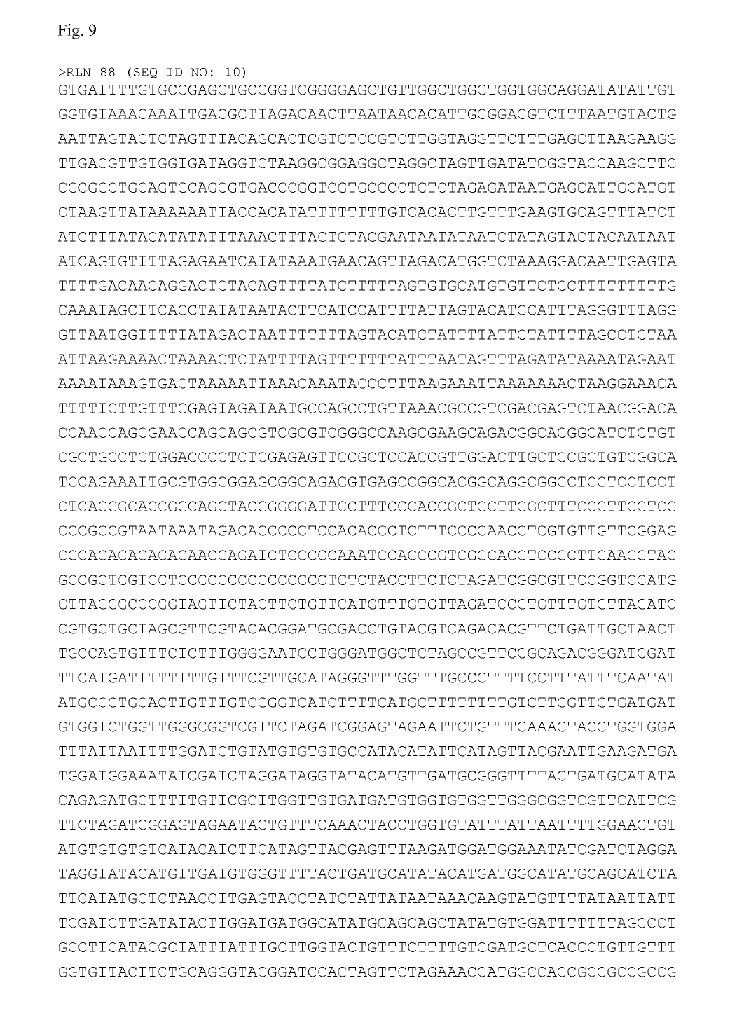

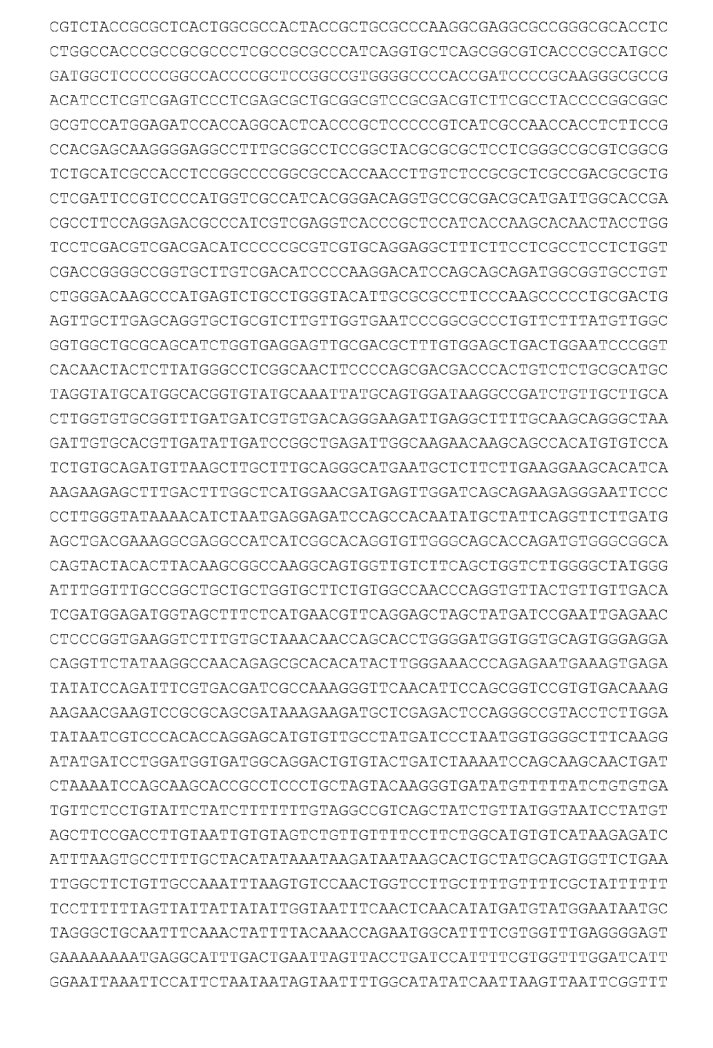

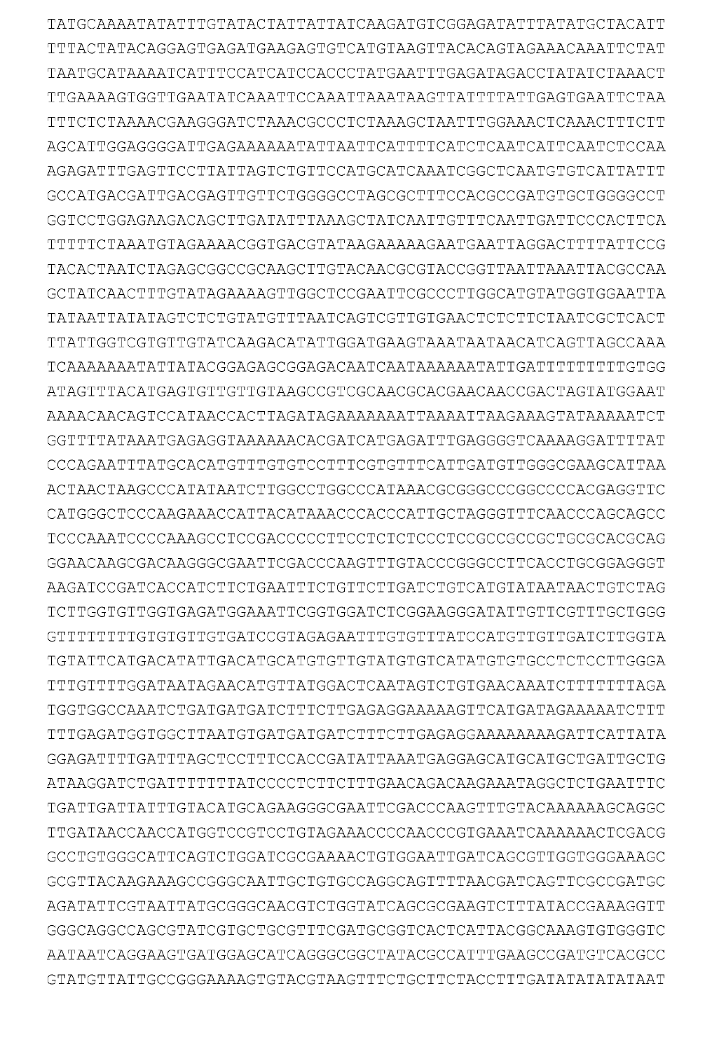

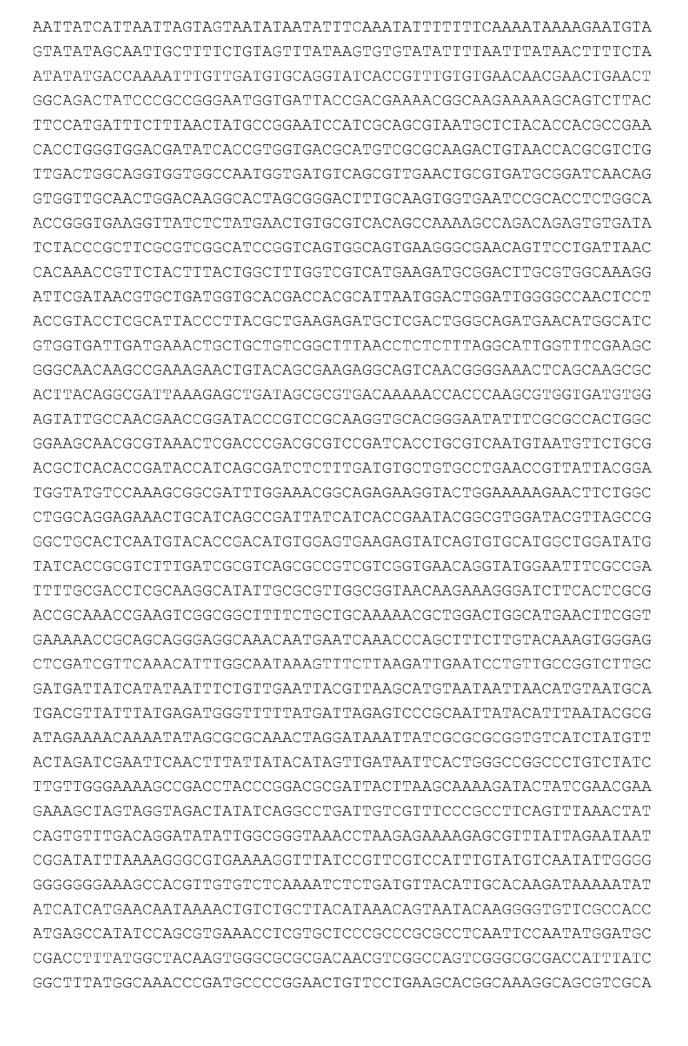

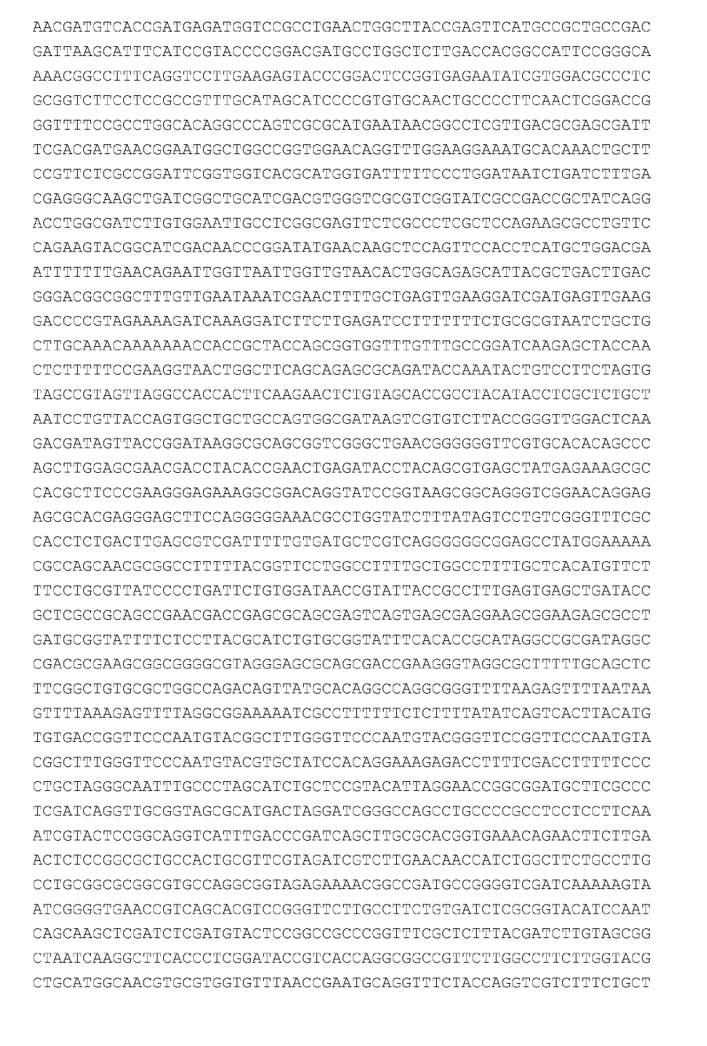

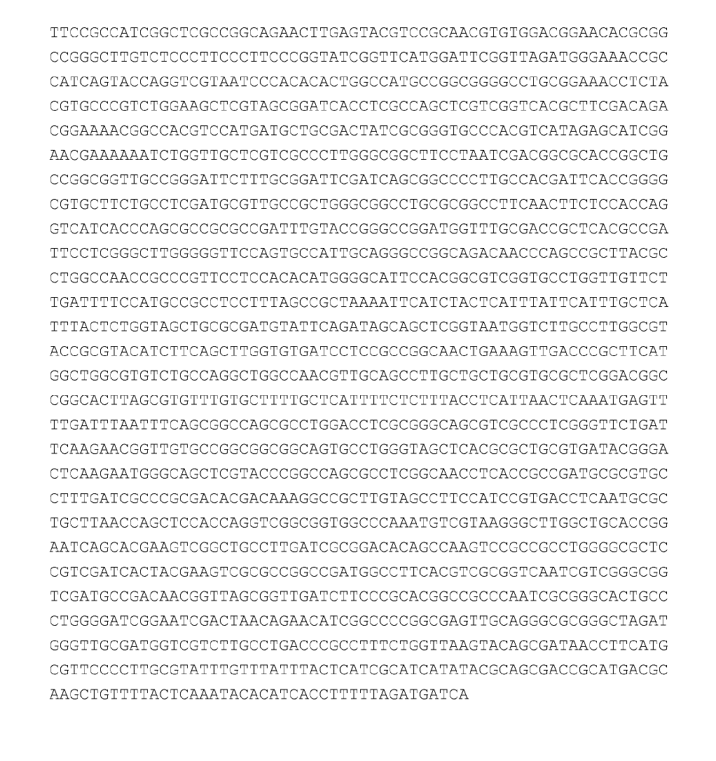

The 651 by promoter fragment amplified by PCR was cloned into pENTR™ 5′-TOPO TA Cloning vector (Invitrogen, Carlsbad, Calif., USA). A BASF Plant Science proprietary intron-mediated enhancement (IME)-intron (BPSI.1) was inserted into the restriction enzyme BsrGl site that is 24 by downstream of the 3′ end of the ZmNP27. The resulting vector was used as a Gateway entry vector in order to produce the final binary vector RLN 88 that has pZmNP27::BPSI.1::GUS::t-NOS cassette for maize transformation (FIG. 8) to characterize the function of pZmNP27 in the forward direction. Sequence of the binary vector RLN 88 is shown in FIG. 9.

### Example 9

### Binary Vector Construction for Maize Transformation to Identify the Function of pZmNP27 in Both Forward and Reverse Directions

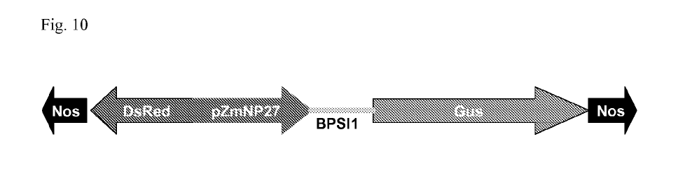

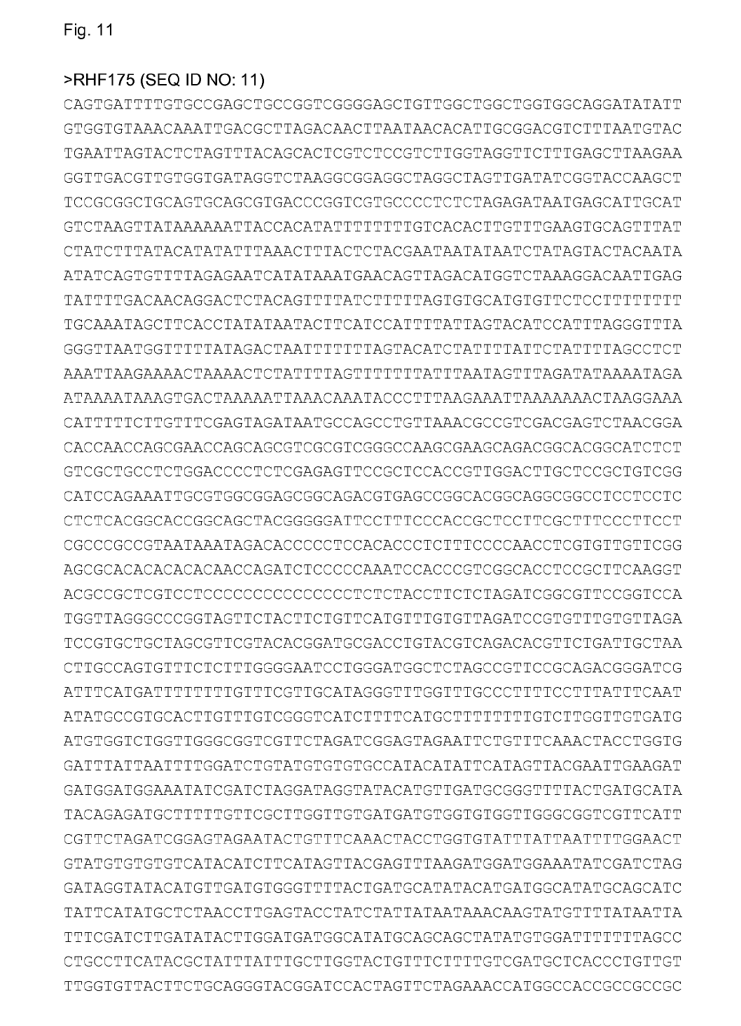

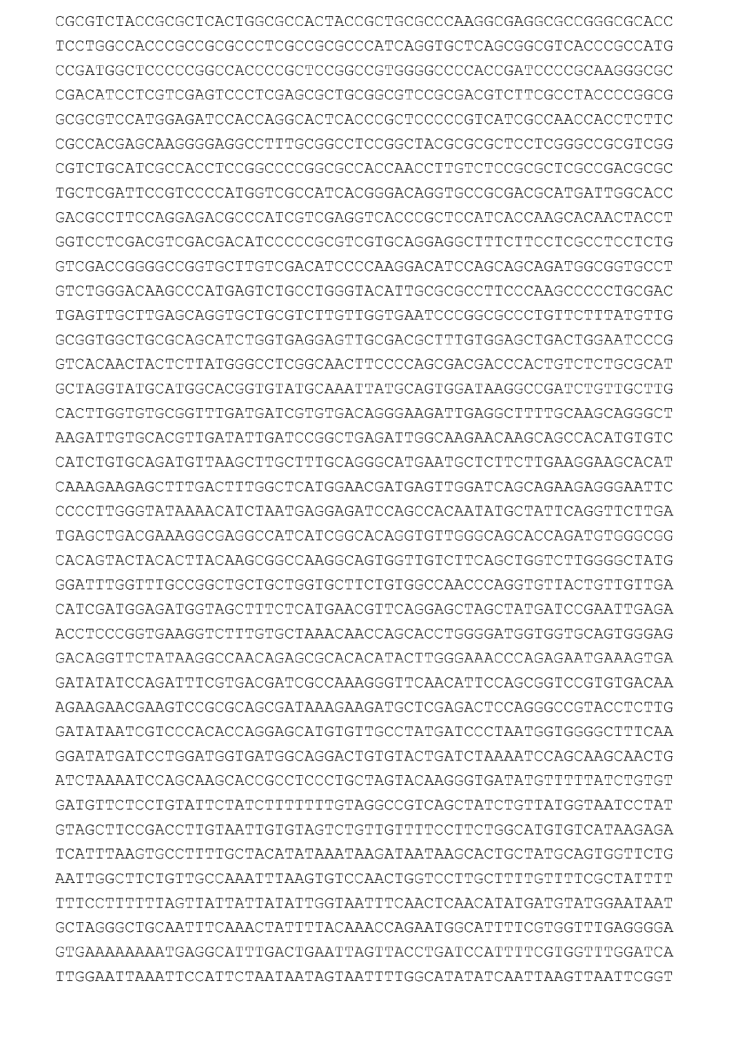

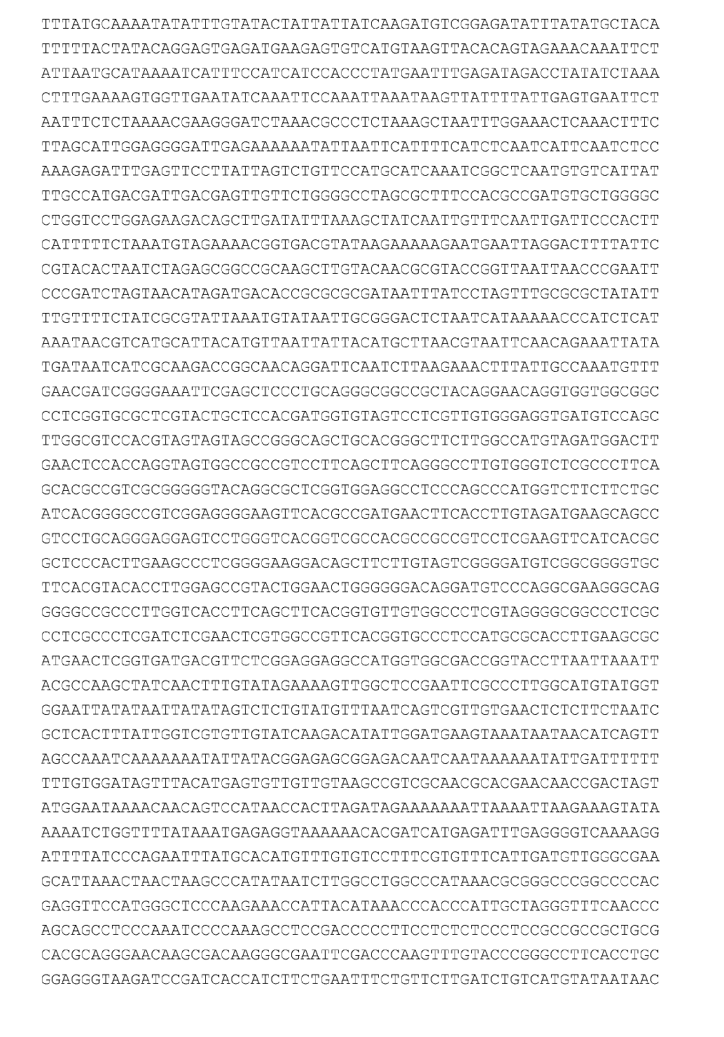

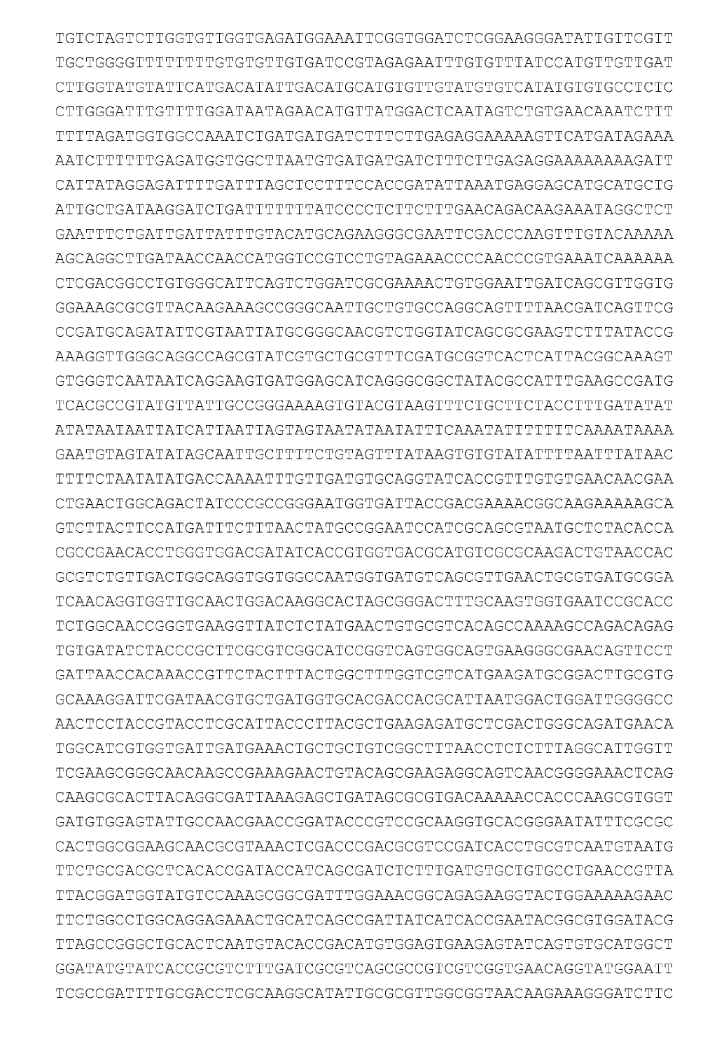

To determine if the pZmNP27 functions bi-directionally, another binary vector, RHF175 was constructed. The GUS reporter gene in combination with the NOS terminator (GUS::NOS) was fused downstream of BPSI.1 intron, which became a construct named RLN88. The GUS gene expression was controlled by the pZmNP27 in forward direction. RLN88 also contains a plant selectable marker cassette between LB and the GUS reporter gene cassette. The second reporter gene, DsRed, in combination with the NOS terminator (DsRed::NOS) was fused upstream of the 5′end of pZmNP27 in RLN88. The expression of this DsRed gene was controlled by the pZmNP27 in reverse direction. The tesulting construct was named RHF175. The reporter gene cassette in RHF175 is structured as follows: t-NOS::DsRed::pZmNP27::BPSI.1::GUS::t-NOS (FIG. 10). The sequence of RHF175 is shown in FIG. 11.

### Example 10

### Promoter Characterization in Transgenic Maize with RLN 88

Expression patterns and levels driven by the ZmNP27 promoter were measured using GUS histochemical analysis following the protocol in the art (Jefferson 1987). Maize transformation was conducted using an Agrobacterium-mediated transformation system. Ten and five single copy events for T0 and T1 plants were chosen for the promoter analysis. GUS expression was measured at various developmental stages:

1) Roots and leaves at 5-leaf stage

2) Stem at V-7 stage

2) Leaves, husk and silk at flowering stage (first emergence of silk)

3) Spikelets/Tassel (at pollination)

5) Ear or Kernels at 5, 10, 15, 20, and 25 days after pollination (DAP)

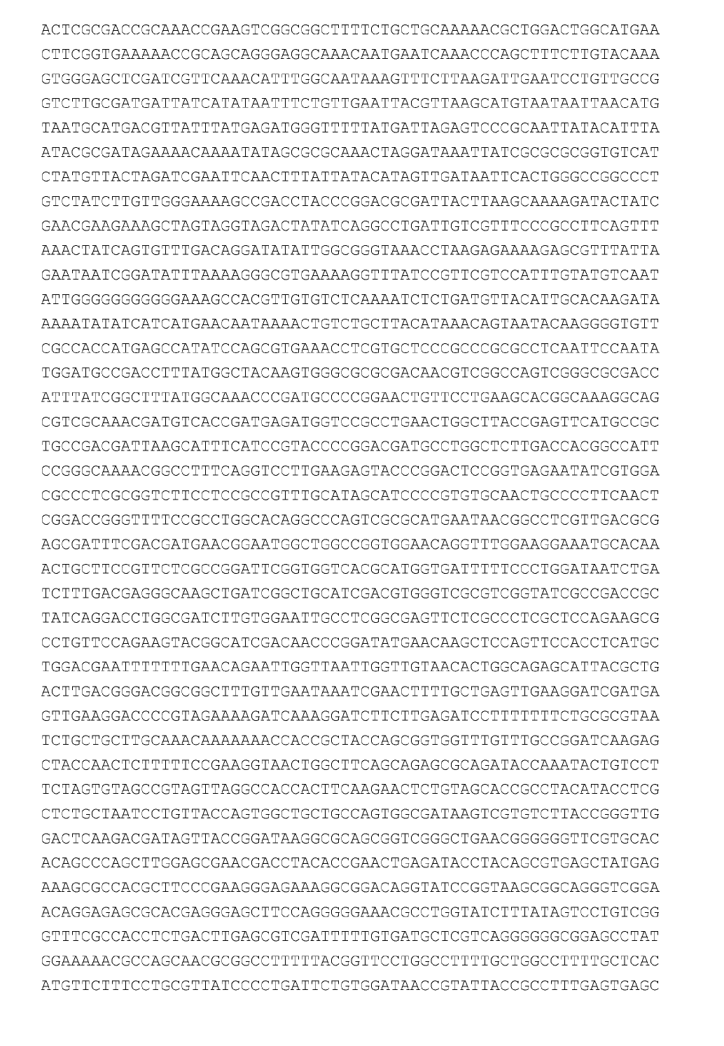

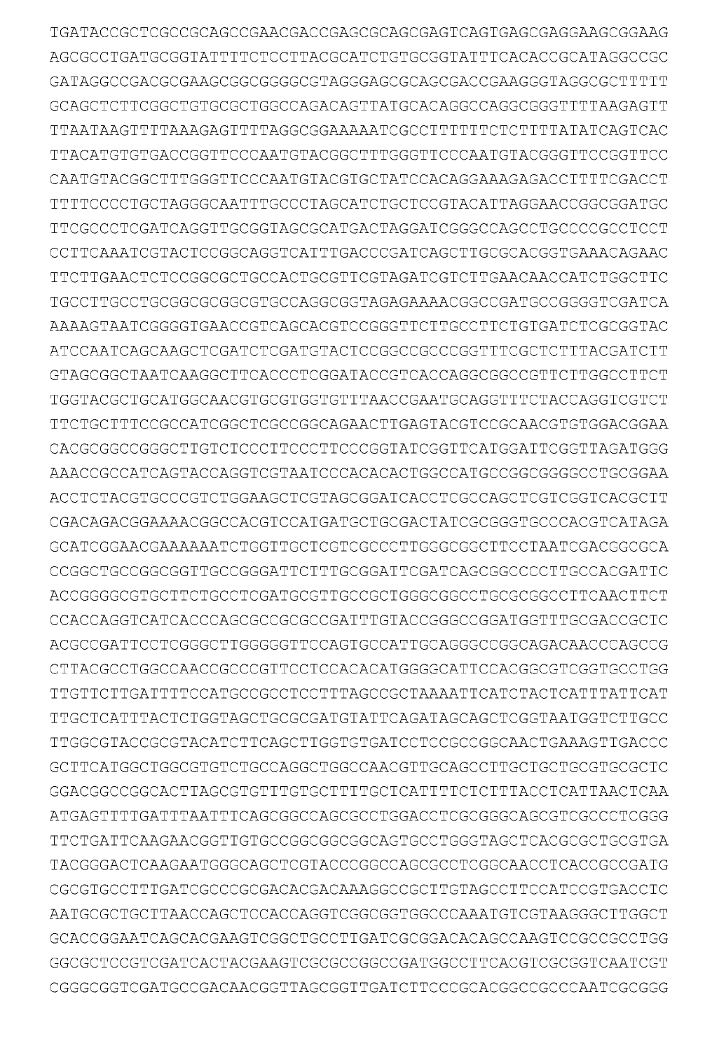

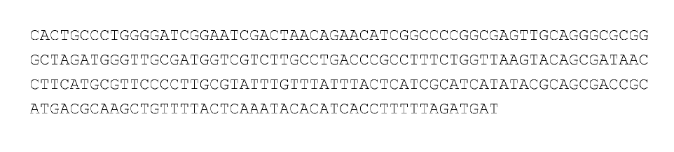

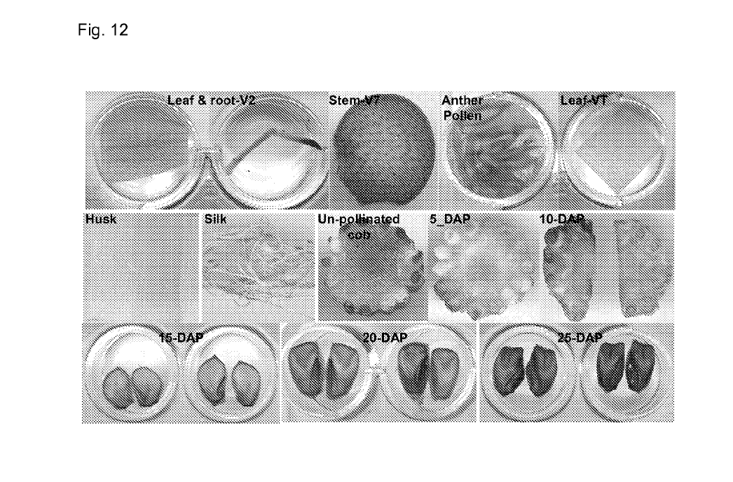

The results indicated that forward direction of ZmNP27 of RHF88 functioned constitutively with preferable expression in whole seeds and stem (FIG. 12).

### Example 11

### Promoter Characterization in Transgenic Maize with RHF175

Expression patterns and levels driven by the ZmNP27 promoter in both directions were measured using GUS histochemical analysis for the GUS reporter as stated above and using fluorescence scanner Typhoon 9400 for DsRed reporter expression. The tissue types and developmental stages were the same as listed above.

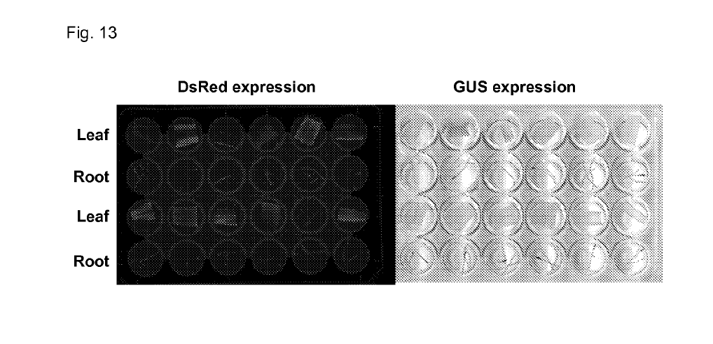

The pZmNP27 in reverse direction expressed DsRed gene in leaf and root but not in Seed (FIG. 13). The pZmNP27 in reverse direction expressed DsRed gene in leaf and root but not in Seed (FIG. 13).

### Example 12

### Deletion Experiment of Promoter ZmNP27 to Identify the Key Regions for Function

Two deletions were made to identify the key regions for the promoter function:

The 159 bp fragment from the 5′ end of pZMNP27 was deleted. The remaining 492 by of the promoter region including the 5′ UTR (FIG. 145) was named pZmNP18. The pZmNP27 in RLN88 was replaced with pZmNP18, which became a construct named RLN87.

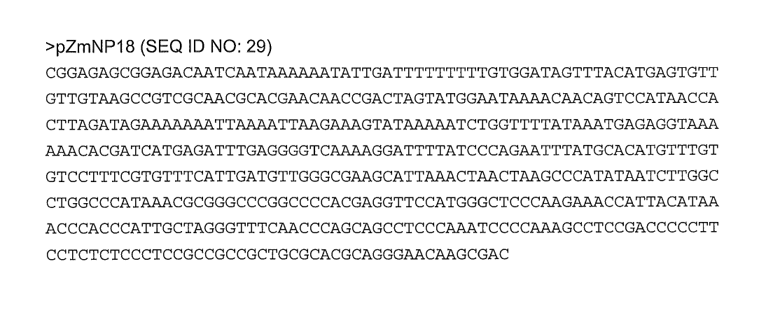

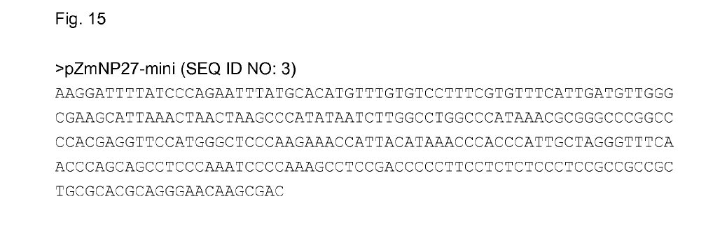

The 380 bp from the 5′ end of pZMNP27 was deleted. The remaining 271 by promoter region including the 5′UTR (FIG. 15) was named pZmNP27-mini. The pZmNP27 in RLN88 was replaced with pZmNP27-mini, which became a construct named RHF178.

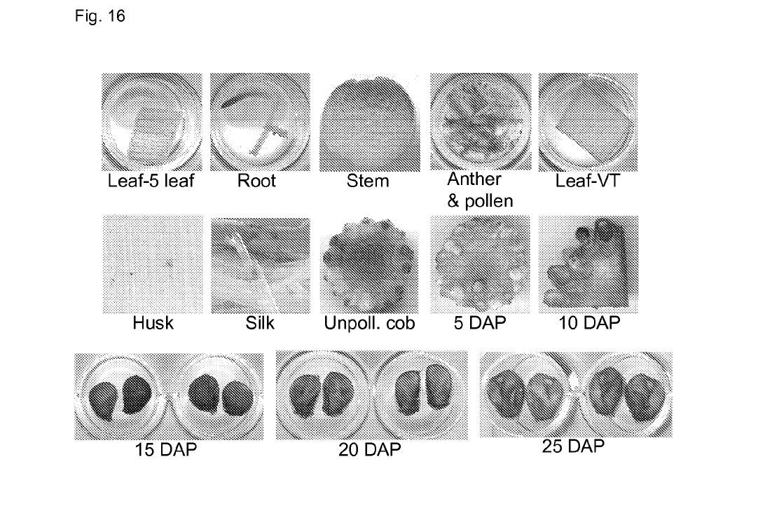

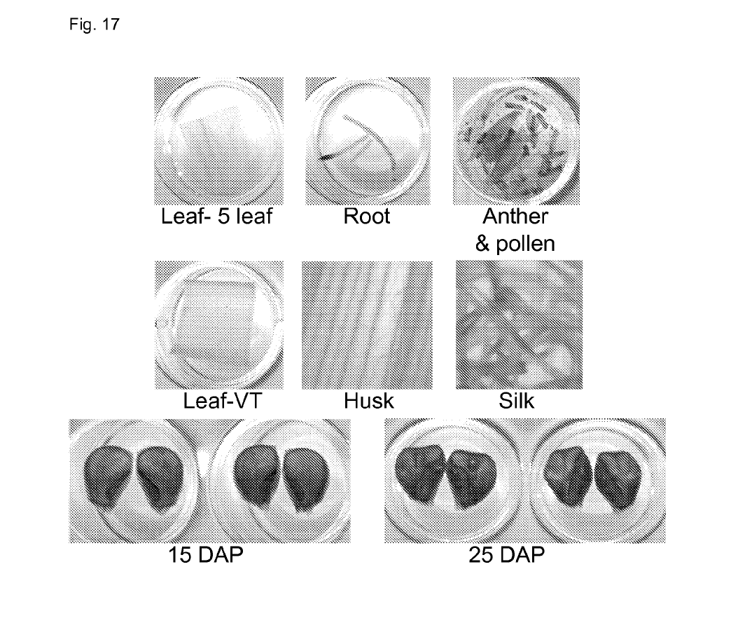

Both pZmNP18 and pZmNP27-mini functioned very similar to the full length of forward pZmNP27 in maize. The expression results in transgenic plant with RLN87 and RHF178 are shown in FIG. 16 and FIG. 17, respectively.

1. A polynucleotide comprising an expression control sequence which allows for bidirectional expression of two nucleic acids of interest being operatively linked thereto in opposite orientations, wherein said expression control sequence being is selected from the group consisting of:
(a) an expression control sequence having a nucleic acid sequence as shown in any one of SEQ ID NOs: 1 to 3;
(b) an expression control sequence having a nucleic acid sequence which is at least 80% identical to a nucleic acid sequence shown in any one of SEQ ID NOs: 1 to 3;
(c) an expression control sequence having a nucleic acid sequence which hybridizes under stringent conditions to a nucleic acid sequence as shown in any one of SEQ ID NOs: 1 to 3;
(d) an expression control sequence having a nucleic acid sequence which hybridizes to a nucleic acid sequence located upstream of an open reading frame sequence shown in SEQ ID NO: 4;
(e) an expression control sequence having a nucleic acid sequence which hybridizes to a nucleic acid sequence located upstream of an open reading frame sequence encoding an amino acid sequence as shown in SEQ ID NO: 5;
(f) an expression control sequence having a nucleic acid sequence which hybridizes to a nucleic acid sequence located upstream of an open reading frame sequence being at least 80% identical to an open reading frame sequence as shown in SEQ ID NO: 4, wherein the open reading frame encodes a 60S acidic ribosomal protein P3;
(g) an expression control sequence having a nucleic acid sequence which hybridizes to a nucleic acid sequence located upstream of an open reading frame encoding an amino acid sequence being at least 80% identical to an amino acid sequence as shown in SEQ ID NO: 5, wherein the open reading frame encodes a 60S acidic ribosomal protein P3;
(h) an expression control sequence obtainable by 5′ genome walking or by thermal asymmetric interlaced polymerase chain reaction (TAIL-PCR) on genomic DNA from the first exon of an open reading frame sequence as shown in SEQ ID NO: 4;
(i) an expression control sequence obtainable by 5′ genome walking or TAIL PCR on genomic DNA from the first exon of an open reading frame sequence being at least 80% identical to an open reading frame as shown in SEQ ID NO: 4, wherein the open reading frame encodes a 60S acidic ribosomal protein P3; and
(j) an expression control sequence obtainable by 5′ genome walking or TAIL PCR on genomic DNA from the first exon of an open reading frame sequence encoding an amino acid sequence being at least 80% identical to an amino acid sequence encoded by an open reading frame as shown in SEQ ID NO: 5, wherein the open reading frame encodes a 60S acidic ribosomal protein P3.

2. The polynucleotide of claim 1, wherein said polynucleotide further comprises at least one nucleic acid of interest being operatively linked to the expression control sequence.

3. The polynucleotide of claim 1 , wherein said polynucleotide further comprises at least one nucleic acid of interest being operatively linked to the expression control sequence in each of the opposite orientations.

4. The polynucleotide of claim 1, wherein said nucleic acid of interest is heterologous with respect to the expression control sequence.

5. A vector comprising the polynucleotide of claim 1.

6. The vector of claim 5, wherein said vector is an expression vector.

7. A host cell comprising the polynucleotide of claim 1 or a vector comprising said polynucleotide.

8. The host cell of claim 7, wherein said host cell is a plant cell.

9. A non-human transgenic organism comprising the polynucleotide of claim 1 or a vector comprising said polynucleotide.

10. The non-human transgenic organism of claim 9, wherein said organism is a plant or a plant seed.

11. A method for expressing a nucleic acid of interest in a host cell comprising:
(a) introducing the polynucleotide of claim 1 or a vector comprising said polynucleotide into a host cell; and
(b) expressing at least one nucleic acid of interest in said non-human transgenic organism.

12. The method of claim 11, wherein said host cell is a plant cell.

13. A method for expressing a nucleic acid of interest in a non-human organism comprising:
(a) introducing the polynucleotide of claim 1 or a vector comprising said polynucleotide into the non-human organism; and
(b) expressing at least one nucleic acid of interest in said non-human transgenic organism.

14. The method of claim 13, wherein said non-human transgenic organism is a plant or seed thereof.

15. The method of claim 13, wherein said at least one nucleic acid of interest is expressed in each orientation from the expression control sequence.

16. (canceled)


SEQUENCE LISTING (29 sequences)

SEQ ID NO: 1 - DNA, 651 bp, Zea mais
ggcatgtatggtggaattatataattatatagtctctgtatgtttaatcagtcgttgtgaactctcttctaatcgctcactttattggtcgtgttgtatcaagacatattggatgaagtaaataataacatcagttagccaaatcaaaaaaatattatacggagagcggagacaatcaataaaaaatattgatttttttttgtggatagtttacatgagtgttgttgtaagccgtcgcaacgcacgaacaaccgactagtatggaataaaacaacagtccataaccacttagatagaaaaaaattaaaattaagaaagtataaaaatctggttttataaatgagaggtaaaaaacacgatcatgagatttgaggggtcaaaaggattttatcccagaatttatgcacatgtttgtgtcctttcgtgtttcattgatgttgggcgaagcattaaactaactaagcccatataatcttggcctggcccataaacgcgggcccggccccacgaggttccatgggctcccaagaaaccattacataaacccacccattgctagggtttcaacccagcagcctcccaaatccccaaagcctccgacccccttcctctctccctccgccgccgctgcgcacgcagggaacaagcgac

SEQ ID NO: 2 - DNA, 651 bp, Zea mais; identical to SEQ ID NO: 1

SEQ ID NO: 3 - DNA, 271 bp, Zea mais
aaggattttatcccagaatttatgcacatgtttgtgtcctttcgtgtttcattgatgttgggcgaagcattaaactaactaagcccatataatcttggcctggcccataaacgcgggcccggccccacgaggttccatgggctcccaagaaaccattacataaacccacccattgctagggtttcaacccagcagcctcccaaatccccaaagcctccgacccccttcctctctccctccgccgccgctgcgcacgcagggaacaagcgac

SEQ ID NO: 4 - DNA, 363 bp, Zea mais
atgggcgtctacacctttgtgtgccgcaacaacggcggcgagtggaccgccaagcagcactccggcgagatcgaggcttccgccgctaccccatacgagctgcagcgccgcctcgtcgctgccgcctccgcggccgactctgcctacggcgtccagtcgtccttctccatggtcacccccagctccgccgtgttccaggtgatcgtcggtgctgtcggtggtggtgcaatgatggtgagcggaggtggcggcggcggtgctgctgcatctggtggtgctgctgccgaggctcccaaggaggagaagaaggaagaggagaaggaagagagcgacgacgacatgggattctccctgttcgactag

SEQ ID NO: 5 - protein, 118 aa, Zea mais
MGVYTFVCRNNGGEWTAQHSGEIEASAATPYEQRRLVAAASAADSRYGVQSSFSMVTPSSAVFQVIVGAVGGGAMMVSGGGGGGAAASGGAAAEAPKEEKKEEEKEESDDDMGFSLFD

SEQ ID NO: 6 - DNA, 521 bp, Tricitum L.
  misc_feature: n=a, t, g or c
ggcctccgccgncacnccctacgacctgcagcgccagctngtctccgccgcctgcgccgaggacaagtccggcggcgtccagtcctccttcaccatggtctcccccaagtccgccatcttccaggtggtcattggtggtgcctctgctggtccaatcggtggtggtggtgctgctggtggtgctgctgcatccggtggagctgccgccgaggcccccaaggctgaggagaagaaggaagaggagaaggaggagagcgaggacgaccttggcttctccctctttgattagaccttctttaccctgttgctcgccagaaatgacatggtcctattgccgcagtaattttnnannccatgcccacaggggttgtgaacctgcctagtttgattgtcaagggatgtatgtgctactttctgttcatgtgagganttggttatgttattctgcagcattgaatatggctttagtcttgccannnnnnannnnnnnnnnanncnnnnnnnnnnnnnnnnnnnnnnnn

SEQ ID NO: 7 - DNA, 686 bp, Zea mais
  misc_feature: n=a, t, g or c
cttgtgttgggcatattctaaaggagtttcattaagatccctcaaaatttctgattngccggatccaccaactatggtcaaggccagcagcctcccaaatcccnaannccnntgnnntngtncctctctccntccgccgccgctgcgcacgcagggaacaagcgacatgggcgtctacacctttgtgtgccgcaacaacggcggcgagtggaccgccaagcagcactccggcgagatcgaggcttccgccgctaccccatacgagctgcagcgccgcctngtcgccgccgcctccgcggccgactctgcctacggcgtccagtcgtccttctccatggtcacccccagctccgccgtgttccaggtgatcgtcggtgctgtcggtggtggtgcaatgatggtgagcggaggtggcggcggcggcggtgctgctgcatctggtggtgctgctgccgaggctcccaaggaggagaagaaggaagaggagaaggaagagagcgacgacgacatgggattctccctgttcgactagagttgtgtgtgtcatctggatctcagaagtggtggtttggtcctgttgccgtagttaattttgctgtcctgtctgtgaacctgaagaactcttggtaattgttacaagcgacctgttccagtttatgtgagaatttgatatgttgtttggaatc

SEQ ID NO: 8 - DNA, 745 bp, Zea mais
caaatccccaaagcctccgacccccttcctctctccctccgccgccgctgcgcacgcagggaacaagcgacatgggcgtctacacctttgtgtgccgcaacaacggcggcgagtggaccgccaagcagcactccggcgagatcgaggcttccgccgctaccccatacgagctgcagcgccgcctcgtcgccgccgcctccgcggccgactctgcctacggcgtccagtcgtccttctccatggtcacccccagctccgccgtgttccaggtgatcgtcggtgctgtcggtggtggtgcaatgatggtgagcggaggtggcggcggcggcggtgctgctgcatctggtggtgctgctgccgaggctcccaaggaggagaagaaggaagaggagaaggaagagagcgacgacgacatgggattctccctgttcgactagagttgtgtgtgtcatctggatctcagaagtggtggtttggtcctgttgccgtagttaattttgctgtcctgtctgtgaacctgaagaactcttggtaattgttacaagcgacctgttccagtttatgtgagaatttgatatgttgtttggaatcatatattctccttgccatttgttgtgattttcggtgccaaaaggttactgtttcctgtaatgataatatgtagctagtacttgttcaagaaattgaacagcaacagtactacttgttcaagaaattgaacagcaacagctaaaaaaaaaaaaaa

SEQ ID NO: 9 - DNA, 880 bp, Zea mais
aactttctcttatttggcatgtatggtggaattatataattatatagtctctgtatgtttaatcagtcgttgtgaactctcttctaatcgctcactttattggtcgtgttgtatcaagacatattggatgaagtaaataataacatcagttagccaaatcaaaaaaatattatacggagagcggagacaatcaataaaaaatattgatttttttttgtggatagtttacatgagtgttgttgtaagccgtcgcaacgcacgaacaaccgactagtatggaataaaacaacagtccataaccacttagatagaaaaaaattaaaattaagaaagtataaaaatctggttttataaatgagaggtaaaaaacacgatcatgagatttgaggggtcaaaaggattttatcccagaatttatgcacatgtttgtgtcctttcgtgtttcattgatgttgggcgaagcattaaactaactaagcccatataatcttggcctggcccataaacgcgggcccggccccacgaggttccatgggctcccaagaaaccattacataaacccacccattgctagggtttcaacccagcagcctcccaaatccccaaagcctccgacccccttcctctctccctccgccgccgctgcgcacgcagggaacaagcgacatgggcgtctacacctttgtgtgccgcaacaacggcggcgagtggaccgccaagcagcactccggcgagatcgaggcttccgccgctaccccatacgagctgcagcgccgcctcgtcgctgccgcctccgcggccgactctgcctacggcgtccagtcgtccttctccatggtcacccccagctccgccgtgttccaggtctgttcgccgacgc

SEQ ID NO: 10 - DNA, 13930 bp, artificial sequence
  vector
gtgattttgtgccgagctgccggtcggggagctgttggctggctggtggcaggatatattgtggtgtaaacaaattgacgcttagacaacttaataacacattgcggacgtctttaatgtactgaattagtactctagtttacagcactcgtctccgtcttggtaggttctttgagcttaagaaggttgacgttgtggtgataggtctaaggcggaggctaggctagttgatatcggtaccaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacggatccactagttctagaaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgatccccgcaagggcgccgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgcggcctccggctacgcgcgctcctcgggccgcgtcggcgtctgcatcgccacctccggccccggcgccaccaaccttgtctccgcgctcgccgacgcgctgctcgattccgtccccatggtcgccatcacgggacaggtgccgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccggggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcccggcgccctgttctttatgttggcggtggctgcgcagcatctggtgaggagttgcgacgctttgtggagctgactggaatcccggtcacaactactcttatgggcctcggcaacttccccagcgacgacccactgtctctgcgcatgctaggtatgcatggcacggtgtatgcaaattatgcagtggataaggccgatctgttgcttgcacttggtgtgcggtttgatgatcgtgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggccaacccaggtgttactgttgttgacatcgatggagatggtagctttctcatgaacgttcaggagctagctatgatccgaattgagaacctcccggtgaaggtctttgtgctaaacaaccagcacctggggatggtggtgcagtgggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctaatggtggggctttcaaggatatgatcctggatggtgatggcaggactgtgtactgatctaaaatccagcaagcaactgatctaaaatccagcaagcaccgcctccctgctagtacaagggtgatatgtttttatctgtgtgatgttctcctgtattctatctttttttgtaggccgtcagctatctgttatggtaatcctatgtagcttccgaccttgtaattgtgtagtctgttgttttccttctggcatgtgtcataagagatcatttaagtgccttttgctacatataaataagataataagcactgctatgcagtggttctgaattggcttctgttgccaaatttaagtgtccaactggtccttgcttttgttttcgctatttttttccttttttagttattattatattggtaatttcaactcaacatatgatgtatggaataatgctagggctgcaatttcaaactattttacaaaccagaatggcattttcgtggtttgaggggagtgaaaaaaaatgaggcatttgactgaattagttacctgatccattttcgtggtttggatcattggaattaaattccattctaataatagtaattttggcatatatcaattaagttaattcggttttatgcaaaatatatttgtatactattattatcaagatgtcggagatatttatatgctacatttttactatacaggagtgagatgaagagtgtcatgtaagttacacagtagaaacaaattctattaatgcataaaatcatttccatcatccaccctatgaatttgagatagacctatatctaaactttgaaaagtggttgaatatcaaattccaaattaaataagttattttattgagtgaattctaatttctctaaaacgaagggatctaaacgccctctaaagctaatttggaaactcaaactttcttagcattggaggggattgagaaaaaatattaattcattttcatctcaatcattcaatctccaaagagatttgagttccttattagtctgttccatgcatcaaatcggctcaatgtgtcattatttgccatgacgattgacgagttgttctggggcctagcgctttccacgccgatgtgctggggcctggtcctggagaagacagcttgatatttaaagctatcaattgtttcaattgattcccacttcatttttctaaatgtagaaaacggtgacgtataagaaaaagaatgaattaggacttttattccgtacactaatctagagcggccgcaagcttgtacaacgcgtaccggttaattaaattacgccaagctatcaactttgtatagaaaagttggctccgaattcgcccttggcatgtatggtggaattatataattatatagtctctgtatgtttaatcagtcgttgtgaactctcttctaatcgctcactttattggtcgtgttgtatcaagacatattggatgaagtaaataataacatcagttagccaaatcaaaaaaatattatacggagagcggagacaatcaataaaaaatattgatttttttttgtggatagtttacatgagtgttgttgtaagccgtcgcaacgcacgaacaaccgactagtatggaataaaacaacagtccataaccacttagatagaaaaaaattaaaattaagaaagtataaaaatctggttttataaatgagaggtaaaaaacacgatcatgagatttgaggggtcaaaaggattttatcccagaatttatgcacatgtttgtgtcctttcgtgtttcattgatgttgggcgaagcattaaactaactaagcccatataatcttggcctggcccataaacgcgggcccggccccacgaggttccatgggctcccaagaaaccattacataaacccacccattgctagggtttcaacccagcagcctcccaaatccccaaagcctccgacccccttcctctctccctccgccgccgctgcgcacgcagggaacaagcgacaagggcgaattcgacccaagtttgtacccgggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattcaactttattatacatagttgataattcactgggccggccctgtctatcttgttgggaaaagccgacctacccggacgcgattacttaagcaaaagatactatcgaacgaagaaagctagtaggtagactatatcaggcctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatca

SEQ ID NO: 11 - DNA, 14927 bp, artificial sequence
  vector
cagtgattttgtgccgagctgccggtcggggagctgttggctggctggtggcaggatatattgtggtgtaaacaaattgacgcttagacaacttaataacacattgcggacgtctttaatgtactgaattagtactctagtttacagcactcgtctccgtcttggtaggttctttgagcttaagaaggttgacgttgtggtgataggtctaaggcggaggctaggctagttgatatcggtaccaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacggatccactagttctagaaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgatccccgcaagggcgccgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgcggcctccggctacgcgcgctcctcgggccgcgtcggcgtctgcatcgccacctccggccccggcgccaccaaccttgtctccgcgctcgccgacgcgctgctcgattccgtccccatggtcgccatcacgggacaggtgccgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccggggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcccggcgccctgttctttatgttggcggtggctgcgcagcatctggtgaggagttgcgacgctttgtggagctgactggaatcccggtcacaactactcttatgggcctcggcaacttccccagcgacgacccactgtctctgcgcatgctaggtatgcatggcacggtgtatgcaaattatgcagtggataaggccgatctgttgcttgcacttggtgtgcggtttgatgatcgtgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggccaacccaggtgttactgttgttgacatcgatggagatggtagctttctcatgaacgttcaggagctagctatgatccgaattgagaacctcccggtgaaggtctttgtgctaaacaaccagcacctggggatggtggtgcagtgggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctaatggtggggctttcaaggatatgatcctggatggtgatggcaggactgtgtactgatctaaaatccagcaagcaactgatctaaaatccagcaagcaccgcctccctgctagtacaagggtgatatgtttttatctgtgtgatgttctcctgtattctatctttttttgtaggccgtcagctatctgttatggtaatcctatgtagcttccgaccttgtaattgtgtagtctgttgttttccttctggcatgtgtcataagagatcatttaagtgccttttgctacatataaataagataataagcactgctatgcagtggttctgaattggcttctgttgccaaatttaagtgtccaactggtccttgcttttgttttcgctatttttttccttttttagttattattatattggtaatttcaactcaacatatgatgtatggaataatgctagggctgcaatttcaaactattttacaaaccagaatggcattttcgtggtttgaggggagtgaaaaaaaatgaggcatttgactgaattagttacctgatccattttcgtggtttggatcattggaattaaattccattctaataatagtaattttggcatatatcaattaagttaattcggttttatgcaaaatatatttgtatactattattatcaagatgtcggagatatttatatgctacatttttactatacaggagtgagatgaagagtgtcatgtaagttacacagtagaaacaaattctattaatgcataaaatcatttccatcatccaccctatgaatttgagatagacctatatctaaactttgaaaagtggttgaatatcaaattccaaattaaataagttattttattgagtgaattctaatttctctaaaacgaagggatctaaacgccctctaaagctaatttggaaactcaaactttcttagcattggaggggattgagaaaaaatattaattcattttcatctcaatcattcaatctccaaagagatttgagttccttattagtctgttccatgcatcaaatcggctcaatgtgtcattatttgccatgacgattgacgagttgttctggggcctagcgctttccacgccgatgtgctggggcctggtcctggagaagacagcttgatatttaaagctatcaattgtttcaattgattcccacttcatttttctaaatgtagaaaacggtgacgtataagaaaaagaatgaattaggacttttattccgtacactaatctagagcggccgcaagcttgtacaacgcgtaccggttaattaacccgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctccctgcagggcggccgctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtaccttaattaaattacgccaagctatcaactttgtatagaaaagttggctccgaattcgcccttggcatgtatggtggaattatataattatatagtctctgtatgtttaatcagtcgttgtgaactctcttctaatcgctcactttattggtcgtgttgtatcaagacatattggatgaagtaaataataacatcagttagccaaatcaaaaaaatattatacggagagcggagacaatcaataaaaaatattgatttttttttgtggatagtttacatgagtgttgttgtaagccgtcgcaacgcacgaacaaccgactagtatggaataaaacaacagtccataaccacttagatagaaaaaaattaaaattaagaaagtataaaaatctggttttataaatgagaggtaaaaaacacgatcatgagatttgaggggtcaaaaggattttatcccagaatttatgcacatgtttgtgtcctttcgtgtttcattgatgttgggcgaagcattaaactaactaagcccatataatcttggcctggcccataaacgcgggcccggccccacgaggttccatgggctcccaagaaaccattacataaacccacccattgctagggtttcaacccagcagcctcccaaatccccaaagcctccgacccccttcctctctccctccgccgccgctgcgcacgcagggaacaagcgacaagggcgaattcgacccaagtttgtacccgggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattcaactttattatacatagttgataattcactgggccggccctgtctatcttgttgggaaaagccgacctacccggacgcgattacttaagcaaaagatactatcgaacgaagaaagctagtaggtagactatatcaggcctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgat

SEQ ID NO: 12 - DNA, 21 bp, artificial sequence
  primer
aacaagcgacatgggcgtcta

SEQ ID NO: 13 - DNA, 20 bp, artificial sequence
  primer
aaggacgactggacgccgta

SEQ ID NO: 14 - DNA, 21 bp, artificial sequence
  primer
cgacatgggcgtctacacctt

SEQ ID NO: 15 - DNA, 20 bp, artificial sequence
  primer
aaggacgactggacgccgta

SEQ ID NO: 16 - DNA, 23 bp, artificial sequence
  primer
gtaaagttcttcctgatctgaat

SEQ ID NO: 17 - DNA, 18 bp, artificial sequence
  primer
tcggaagcagccttaata

SEQ ID NO: 18 - DNA, 20 bp, artificial sequence
  primer
ggcatgtatggtggaattat

SEQ ID NO: 19 - DNA, 20 bp, artificial sequence
  primer
gtcgcttgttccctgcgtgc

SEQ ID NO: 20 - DNA, 11 bp, artificial sequence
  consensus sequence (Seq 31)
tggctaactga

SEQ ID NO: 21 - DNA, 15 bp, artificial sequence
  consensus sequence (Seq 51)
tttgacccctcaaat

SEQ ID NO: 22 - DNA, 11 bp, artificial sequence
  consensus sequence (Seq 61)
tgggcttagtt

SEQ ID NO: 23 - DNA, 11 bp, artificial sequence
  consensus sequence (Seq 62)
tgggccaggcc

SEQ ID NO: 24 - DNA, 11 bp, artificial sequence
  consensus sequence (Seq 63)
tggcccataaa

SEQ ID NO: 25 - DNA, 11 bp, artificial sequence
  consensus sequence (Seq 64)
cgggcccgcgt

SEQ ID NO: 26 - DNA, 11 bp, artificial sequence
  consensus sequence (Seq 67)
cgggcccggcc

SEQ ID NO: 27 - DNA, 11 bp, artificial sequence
  consensus sequence (Seq 68)
ggggccgggcc

SEQ ID NO: 28 - DNA, 11 bp, artificial sequence
  consensus sequence (Seq 69)
tgggctcccaa

SEQ ID NO: 29 - DNA, 492 bp, Zea mais
cggagagcggagacaatcaataaaaaatattgatttttttttgtggatagtttacatgagtgttgttgtaagccgtcgcaacgcacgaacaaccgactagtatggaataaaacaacagtccataaccacttagatagaaaaaaattaaaattaagaaagtataaaaatctggttttataaatgagaggtaaaaaacacgatcatgagatttgaggggtcaaaaggattttatcccagaatttatgcacatgtttgtgtcctttcgtgtttcattgatgttgggcgaagcattaaactaactaagcccatataatcttggcctggcccataaacgcgggcccggccccacgaggttccatgggctcccaagaaaccattacataaacccacccattgctagggtttcaacccagcagcctcccaaatccccaaagcctccgacccccttcctctctccctccgccgccgctgcgcacgcagggaacaagcgac